(19) United States
(12) Patent Application Publication
(10) Pub. No.: US 2015/0159115 A1
(43) Pub. Date: Jun. 11, 2015

(54) PURIFICATION OF DPA ENRICHED OIL

(71) Applicant: Omega Protein Corporation, Houston, TX (US)

(72) Inventors: Oleksandr A. Byelashov, Houston, TX (US); Huaixia Yin, Houston, TX (US); Mark E. Griffin, Houston, TX (US)

(21) Appl. No.: 14/532,893

(22) Filed: Nov. 4, 2014

Related U.S. Application Data
(60) Provisional application No. 61/902,055, filed on Nov. 8, 2013.
(60) Provisional application No. 61/902,045, filed on Nov. 8, 2013.
(60) Provisional application No. 61/975,694, filed on Apr. 4, 2014.

(51) Int. Cl. C11B 3/10; B01D 15/32; A61K 31/202

(52) U.S. Cl. CPC C11B 3/10; A61K 31/202; B01D 15/325

(57) ABSTRACT

The present invention provides chromatographic means for separating from a starting lipid mixture a mixture that is enriched in docosapentaenoic acid relative to the starting lipid mixture. The invention also provides pharmaceutical formulations and dietary supplements including this enriched lipid formulation, and methods of supplementing docosapentaenoic acid in a subject by administering to the subject the pharmaceutical formulation or the dietary supplement. The invention also provides a method of resolving from the starting lipid mixture a mixture enriched in docosahexaenoic acid and eicosapentaenoic acid and depleted in docosapentaenoic acid, and formulations including this mixture (e.g., pharmaceutical formulations and dietary supplements). Also provided are methods of supplementing docosapentaenoic acid and eicosapentaenoic acid levels in a subject by administering a formulation of the invention to the subject. In various embodiments, the formulations of the invention are of use to lower cholesterol, triglyceride, glucose levels, inflammation, in the subject to whom the formulation is administered.

### CROSS-REFERENCE TO RELATED APPLICATIONS

This application claims priority to U.S. Provisional Patent Application No. 61/902,045 filed Nov. 8, 2013, U.S. Provisional Patent Application No. 61/902,055 filed Nov. 8, 2013 and U.S. Provisional Patent Application No. 61/975,694 filed Apr. 4, 2014, the disclosures of which are incorporated herein by reference in their entirety for all purposes.

### FIELD OF THE INVENTION

The invention resides in preparation of formulations of polyunsaturated fatty acids and the use of these formulations to treat various conditions in subjects needing such treatment.

### BACKGROUND OF THE INVENTION

The American Heart Association estimates that more than 1 in 3 Americans have cardiovascular disease (CVD). Cardiovascular disease kills more Americans per year than cancer, averaging more than 2200 deaths per day. Atherosclerosis, which is the greatest contributor to CVD, is caused by a build-up of lipids, cholesterol, and apoptotic bodies in the intima of the arterial wall. These build-ups are called plaques, which cause a narrowing of the lumen of the artery and a fibrous cap is formed on the endothelial layer of the artery. The narrowing of the lumen can cause a blockage if platelets accumulate in the area, and if the plaque gets large enough eventually the fibrous cap will burst, causing a thrombosis. The annual cost for CVD is estimated at $297.7 billion which includes $118.5 billion in lost future productivity due to premature CVD mortality. This is attributed to fewer than half of the people that have experienced a cardiac event or are qualified to receive a lipid lowering drug receiving treatment (Roger, et al., 2012. Circulation, 125:e2-e220).

Recommendations for the consumption of omega-3 fatty acids by the United States Department of Agriculture have only been determined for linolenic acid (C18:3n3). The American Heart Association, recently recommended that the daily consumption of eicosapentaenoic acid (EPA)+docosahexaenoic acid (DHA) for individuals with differing levels of fasting triacylglycerol should be 0.5-1.0 g (borderline, 150-199 mg/dL), 1-2 g (high, 200-499 mg/dL), and 2-4 g (very high, >500 mg/dL; Miller, et al., 2011, Circulation. 123:2292-333). The American Heart Association estimates that only 10-15% of Americans consume the recommended amount (≧250 mg per day) of very-long chain polyunsaturated fatty acids (PUFAs), specifically EPA and DHA, and 15-40% of Americans consume the recommended amount of nuts, legumes and seeds (Roger, et al., 2012, Circulation, 125:e2-e220).

Docosapentaenoic acid n-3 (DPAn3) is formed from the elongation of EPA, which can then be elongated, desaturated, transported, then shortened to DHA (Kaur, et al., 2011, Prog. Lipid. Res., 50:28-34). Arachidonic acid is elongated and desaturated to DPAn6 which physiologically acts very different from DPAn3. DPAn6 does not provide the protective effect that DHA does to neural cells for survival and prevention of apoptosis (Kim, et al., 2003, Lipids., 38:453-457). Docosapentaenoic acid n-3 upregulates lipooxygenase (LOX) pathway and also acts as a potent inhibitor of cyclooxygenase-1 (COX-1) and COX-2 activity leading to decreased platelet aggregation and active tension of the aorta (Akiba, et al., 2000, Biol. Pharm. Bull., 23:1293-1297; Chen, et al., 2012, Atherosclerosis., 221:397-404). Mice fed a diet supplemented with pure DPAn-3 had decreased fatty acid synthase activity, total cholesterol, and TAG levels (Gotoh, et al., 2009, J. Agric. Food Chem., 57:11047-11054). When hamsters were switched from a normal chow diet to a high-cholesterol diet, DPAn3 supplementation prevented an increase in TC, which the researches attributed to a down-regulation of SREBP-2 subsequently decreasing transcription of HMG-CoA reductase, involved in cholesterol synthesis (Chen, et al., 2012, Atherosclerosis, 221:397-404).

Post prandial plasma chylomicronemia has been found to be decreased in humans following a breakfast supplemented with DPAn3, and DPAn3 was found to be transported in the chylomicron TAG rather than the chylomicron phospholipids over the 5 hour period followed (Linderborg, et al., 2013, Prostaglandins. Leukot. Essent. Fatty Acids, 88:313-319). This means that the enterocyte is choosing to esterify free fatty acids to a glycerol backbone to make TAG instead of esterifying to a lysophospholipid to make a phospholipid when assembling the chylomicron. The presence of DPAn3 also decreased digestion, absorption or incorporation into a chylomicron of the olive oil present in the breakfast. The incorporation of DPAn3 into adipose, heart, and skeletal muscle tissue is very high (Kaur, et al., 2010; Kaur, et al., 2013, Br. J. Nutr., 109:441-448). A change in cortical tissue concentration of n3 FAs is dependent upon feeding. Regardless of age oral administration of DPAn3 to rats led to an increase of DPAn3 and DHA present in cortical tissue (Kelly, et al., 2011, Neurobiol Aging., 32:2318.e1-2318.e15). Murine resident peritoneal macrophages preferentially incorporate DPAn3 into their membranes, even when EPA is supplemented; this is due to EPA rapidly elongating into DPAn3 (˜75%). When cells were stimulated with ATP, DPAn3 was the only PUFA released at a level comparable to AA release and decreased the production of eicosanoids such as 11-hydroxy-5,8,12,14-eicosatetraenoic acid and prostaglandin (PG) D₂ (Norris, et al., 2012, PNAS, 109:8517-8522). Recent studies have found a non-linear, inverse relationship between DPAn3 consumption and risk of heart failure, with plasma EPA and DHA having no association with heart failure (Wilk, et al., 2012, Am. J. Clin. Nutr., 96:882-888). Circulating levels of DPAn3 correlate poorly with fish consumption due to the majority of DPAn3 being metabolized from EPA endogenously, as EPA is present in greater levels in fish (Sun, et al., 2008, Am J Clin Nutr., 88:216-23). Questions still remain about the function of DPAn3 in the body and research is becoming more focused on the biological activity and potential of DPAn3.

A greater understanding of the properties and effects of individual ω-3 polyunsaturated acids and mixtures including various ω-3 polyunsaturated acids in controllable proportions would be considerably advanced by a convenient method for separating mixtures of ω-3 polyunsaturated acids into their constituents. The present invention provides such a method and also provides methods of using the compositions prepared by the method to treat various diseases.

### BRIEF SUMMARY OF THE INVENTION

In an exemplary embodiment, the present invention provides a chromatographic method of resolving a lipid mixture of ω-3 polyunsaturated acids comprising eicosapentaenoic acid (C20:5n3), docosahexaenoic acid (C22:6n3) and docosapentaenoic acid (C22:5n3) into a first and second fraction. An exemplary first fraction comprises docosapentaenoic acid of purity from about 20% to about 90%. An exemplary second fraction comprises a mixture of eicosapentaenoic acid (C20:5n3) in about 0% to about 90%, and docosahexaenoic acid (C22:6n3) in about 0% to about 90%. The method includes submitting the lipid mixture to a reverse-phase chromatographic separation under conditions in which two or more components of the lipid mixture are resolved and separated.

In various embodiments, the invention also provides pharmaceutical formulations and formulations for dietary supplements, food additives and the like. Exemplary formulations comprise the first fraction prepared by the method of the invention. In various embodiments, the invention provides such formulations comprising the second fraction.

In exemplary embodiments, the invention provides methods of preventing, treating or otherwise ameliorating diseases, e.g., atherosclerosis, diabetes, metabolic syndrome, inflammation, cognitive decline and neurodegenerative diseases.

Other embodiments, objects and advantages of the invention are apparent from the detailed description that follows.

### BRIEF DESCRIPTION OF THE DRAWINGS

FIG. 1. Mouse serum total cholesterol concentrations after 10 and 20 weeks of an atherogenic diet, supplemented with milk fat (control) or omega-3 fatty acids, alone or in combination, in LDLr−/− mice (Data reported as LSmeans±SEM). Treatment x time P=0.29; treatment P<0.28; time P<0.001; sliced treatment effect by time at week 20 P<0.05. Bars with different superscripts differ within time P<0.10. Open black box across time points indicates 95% confidence interval for baseline value (191±29.2 mg/dL; mean±margin of error). n3 indicates contrast of control diet vs. all omega-3 fatty acid diets at week 20, P<0.05.

FIG. 2. Mouse serum low density lipoprotein (LDL) cholesterol levels after 10 and 20 weeks of an atherogenic feeding protocol, supplemented with milk fat (control) or omega-3 fatty acids, alone or in combination, in LDLr−/− mice (LSmeans±SEM). Treatment x time P=0.24; treatment P=0.19; time P<0.05; sliced treatment effect by time at week 20 P<0.10. Bars with different superscripts differ within time P<0.1. Black box across time points indicates 95% confidence interval for baseline value (131.20±15.13 mg/dL; mean±margin of error).

FIG. 3. Mouse serum triglyceride levels after 10 and 20 weeks of an atherogenic feeding protocol, supplemented with milk fat (control) or omega-3 fatty acids, alone or in combination, in LDLr−/− mice (LSmeans±SEM). Treatment x time P=0.60; treatment P<0.05; time P<0.10; sliced treatment effect by time for week 20 P<0.01. Bars with different superscripts differ within time P<0.1. Black box across time points indicates 95% confidence interval for baseline value (129.92±14.57 mg/dL; mean±margin of error). n3 indicates contrast of control diet vs. all omega-3 fatty acid diets at week 20 P<0.001.

FIG. 4. Mouse serum non-esterified fatty acid (NEFA) levels after 10 and 20 weeks of an atherogenic feeding protocol, supplemented with milk fat (control) or omega-3 fatty acids, alone or in combination, in LDLr−/− mice (LSmeans±SEM). Treatment x time P=0.62; treatment P<0.001; time P<0.01; sliced treatment effect by time for week 10 P<0.01. Bars with different superscripts differ within time P<0.1. Black box across time points indicates 95% confidence interval for baseline value (2569.31±306.50 mg/dL; mean±margin of error). n3 indicates contrast of control diet vs. all omega-3 fatty acid diets at weeks 10 P<0.001 and 20 P<0.05.

FIG. 5. Mouse serum glucose levels after 10 and 20 weeks of an atherogenic feeding protocol, supplemented with milk fat (control) or omega-3 fatty acids, alone or in combination, in LDLr−/− mice (LSmeans±SEM). Treatment x time P=0.16; treatment P=0.22; time P<0.001. Bars with different superscripts differ within time P<0.1. Black box across time points indicates 95% confidence interval for baseline value (74.84±7.73 mg/dL; mean±margin of error).

FIG. 6. Cholesterol levels for total (TC), free (FC), and cholesterol ester (CE) in aorta's of LDLr−/− mice fed an atherogentic feeding protocol, supplemented with milk fat (control) or omega-3 fatty acids, alone or in combination (LSmeans±SEM). Bars with different superscripts differ within time P<0.1. n3 indicates contrast of control diet vs. all omega-3 fatty acid diets for TC P=0.10, and CE P<0.05.

FIG. 7. Concentration of cytokines (tumor necrosis factor alpha (TNF-α), monocyte chemoattractant protein-1 (MCP-1), interleukin-6 (IL-6)) secreted by THP-1 cells into media following two incubations (1: 24 hour incubation with a control, and 11 individual fatty acids at two concentrations (50 & 100 μM), 2: 24 hour stimulation with lipo-polysaccharide (LPS) at three concentrations (averaged across concentrations)). Column means with different superscripts differ within fatty acid concentration P<0.1.

FIG. 8. Chromatography of Typical 300 mg/mL Injected ΩmegaActiv® DPA 5000 Sample.

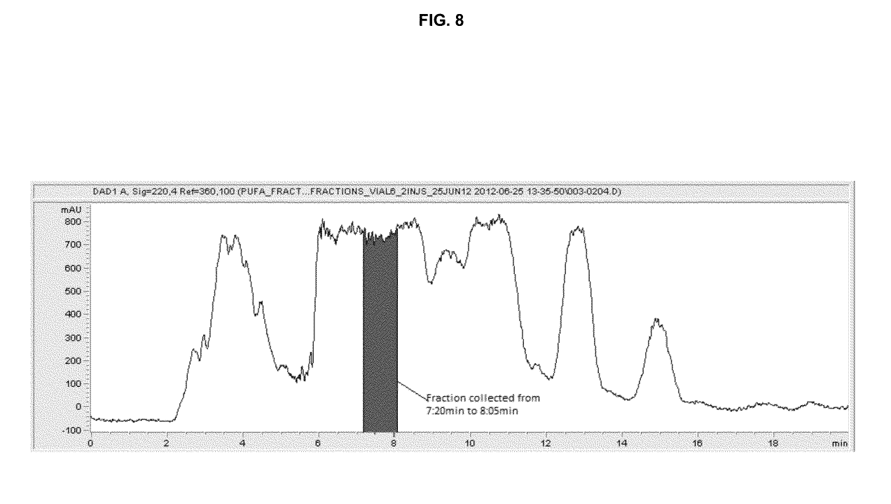

FIG. 9. DPA 0.1 mg/mL Standard (>99% purity).

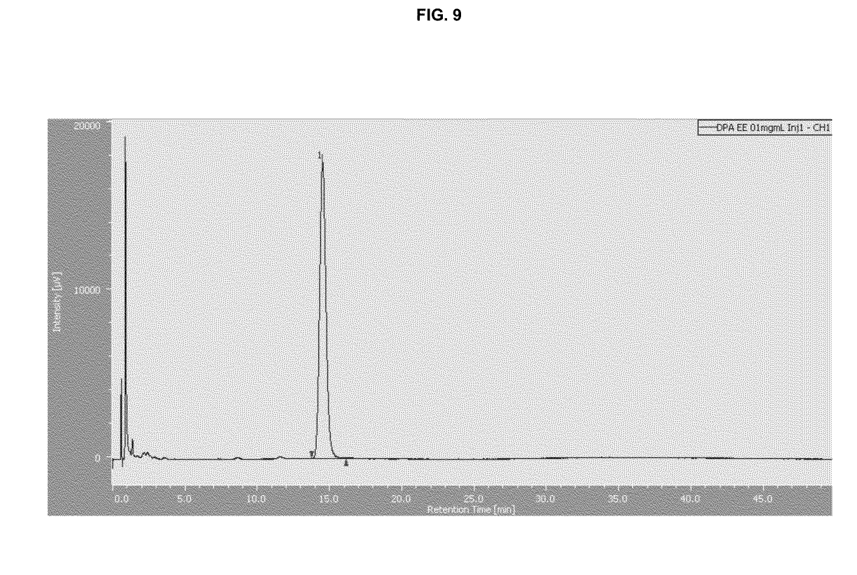

FIG. 10. Starting Material 1.0 mg/mL.

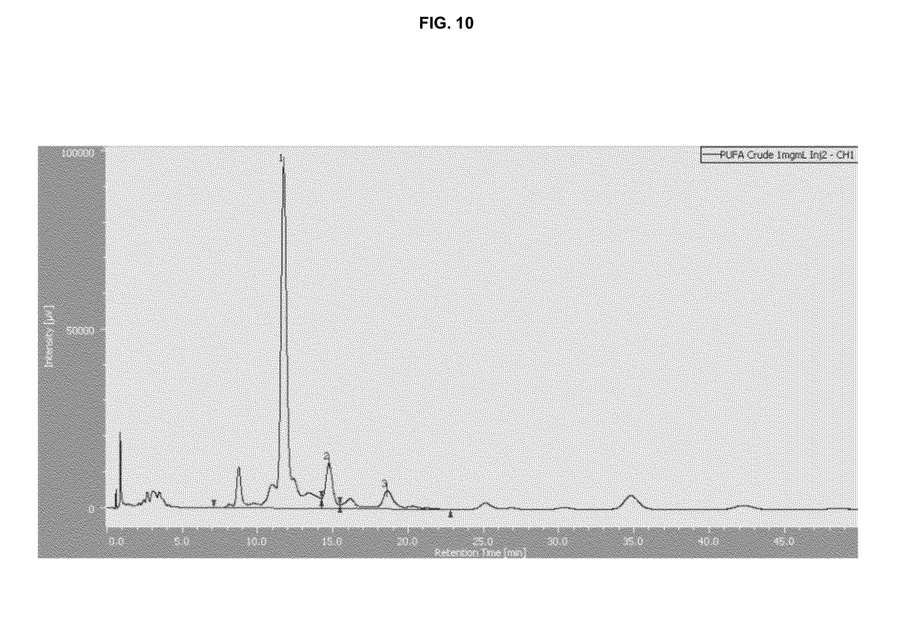

FIG. 11. Final DPA Fraction 0.1 mg/mL=Purified Material.

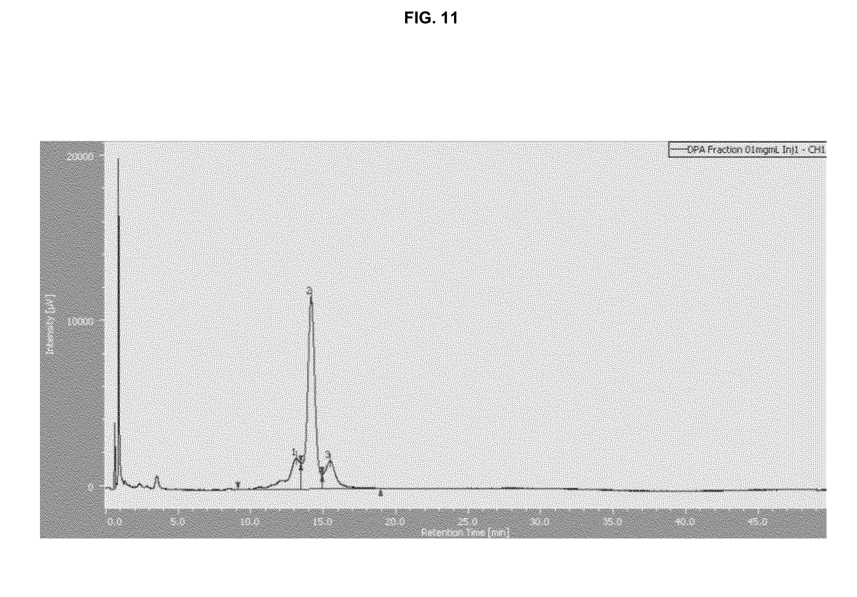

FIG. 12. Chromatography of Typical Injected ΩmegaActiv® DPA 5000 Sample (no dilution).

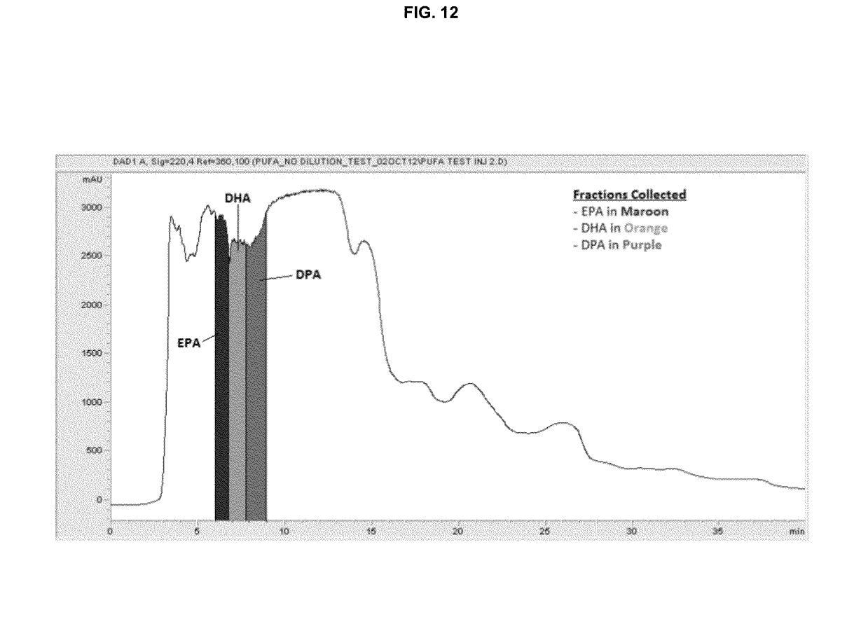

FIG. 13. DPA 0.1 mg/mL Standard.

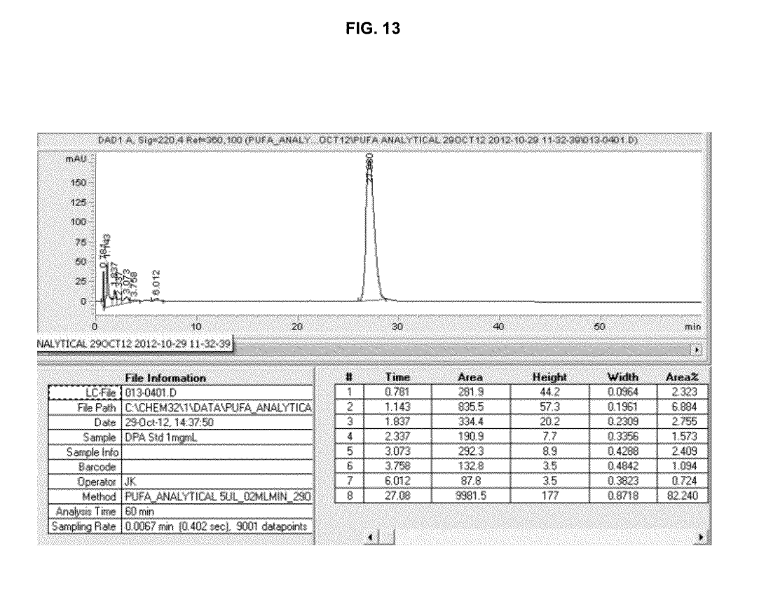

FIG. 14. Final DPA Fraction 1.0 mg/mL=Purified Material.

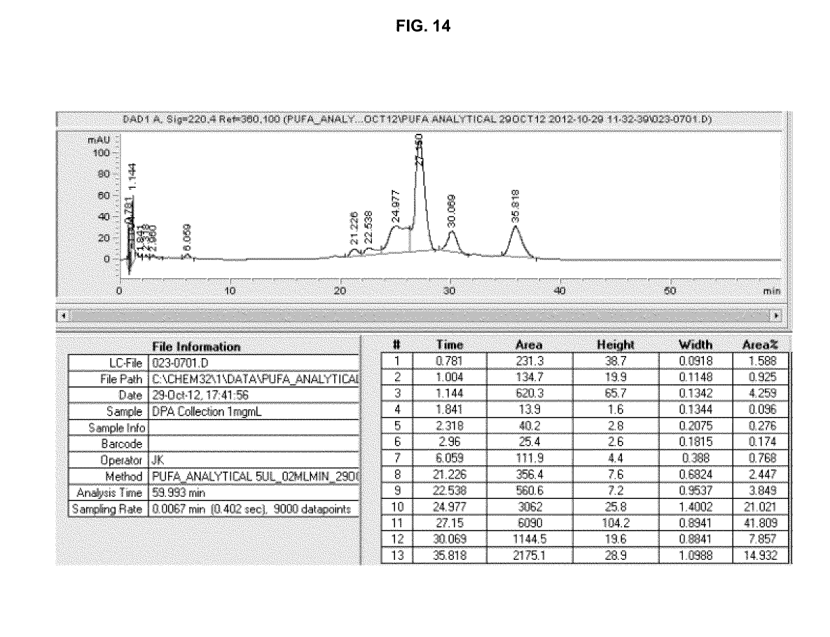

FIG. 15. EPA 0.1 mg/mL Standard.

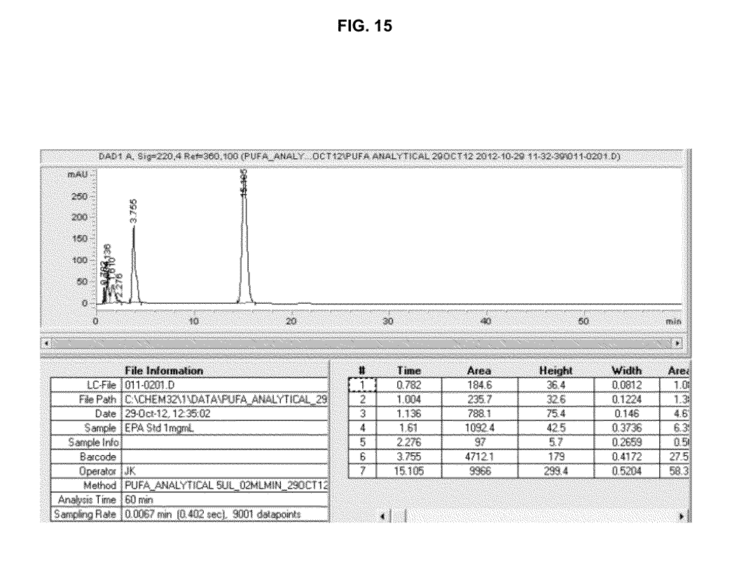

FIG. 16. Final EPA Fraction 1.0 mg/mL=Purified Material.

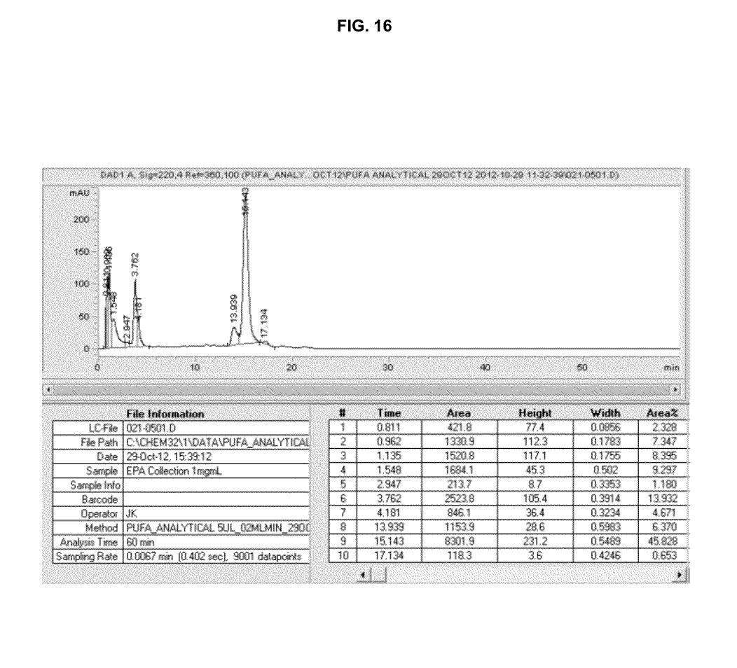

FIG. 17. DHA 0.1 mg/mL Standard.

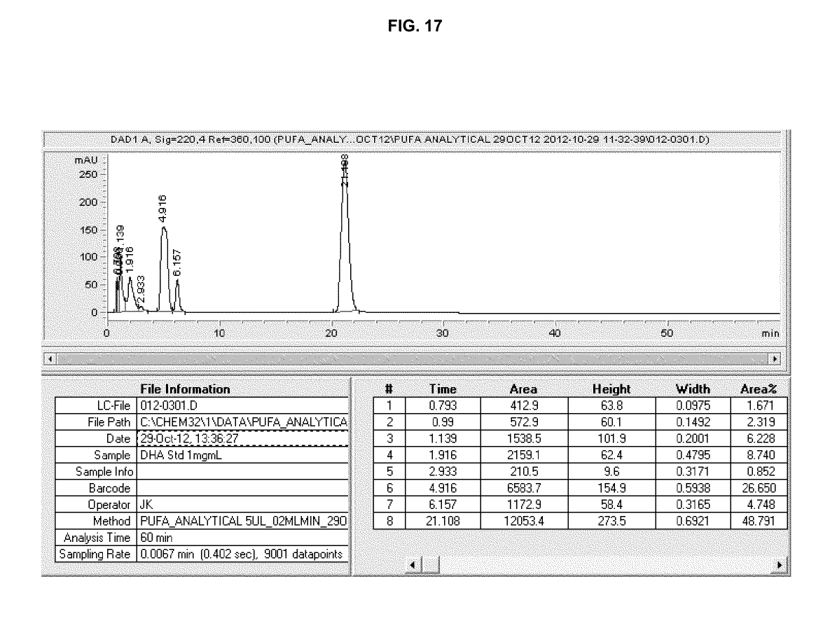

FIG. 18. Final DHA Fraction 1.0 mg/mL=Purified Material.

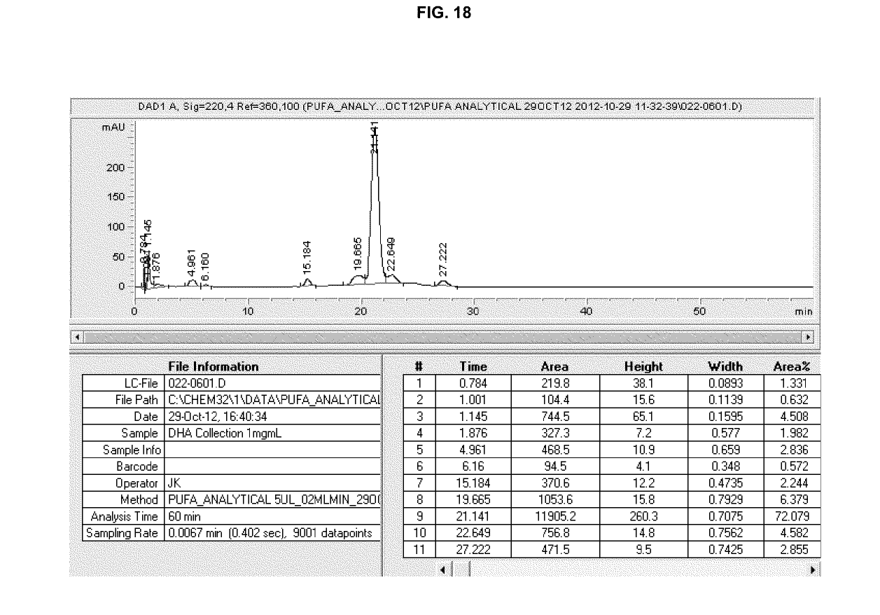

FIG. 19A. Total oxidation of PUFA ethyl ester concentrates (non-accelerated storage 25° C.).

FIG. 19B. Total oxidation of PUFA ethyl ester concentrates (accelerated storage 40° C.).

### DETAILED DESCRIPTION OF THE INVENTION

### I. Introduction

The ω-3 polyunsaturated fatty acids (PUFA) in fish oils such as eicosapentaenoic acid (C20:5n3, EPA) and docosahexaenoic acid (C22:6n3, DHA) have received attention in the scientific and industrial areas because of their positive role in human health including reducing risk of cardiovascular diseases, hypertension and atherosclerosis, inflammatory and autoimmune disorders (Wanasundara, et al., 1998, Journal of American Oil Chemists' Society, 75(8):945-951; Uauy, et al., 2000, Nutrition, 6(7/8):680-684; Horrocks, et al., 1999, Pharmacological Research, 40:211-225; and Benatti, et al., 2004, Journal of the American College of Nutrition, 23:281-302). Biochemical and biological studies on the role of the individual fatty acid requires the development of a practical method for the separation of a certain fatty acid from fish lipid mixtures. Most of these arts were focused on EPA and DHA ω-3 fatty acids. DPA (C22:5n3, docosapentaenoic acid) as another important ω-3 fatty acid is currently of great interest because of evidence that point to beneficial effects on cardiovascular health (Chen, et al., 2012, Atherosclerosis, 221:397-404) and neurological function (Mitchell, et al., 2012, Chemistry and Physics of Lipids, 165:393-400). There's a demand to produce pure DPA to study the health benefits of the individual fatty acids, however, no report was found on how to separate DPA from a mix of fatty acids. PUFA (polyunsaturated fatty acid) concentrates are typically obtained using distillation. However, in contrast to the art-recognized distillation methods, the present invention provides a chromatographic method for the production of highly purified fatty acid fractions. In various embodiments, the invention provides industrial scale high pressure liquid chromatography (HPLC) for PUFA production. The highly purified PUFA formulations, and mixtures of such formulations, are of use for pharmaceuticals and dietary supplements.

In various embodiments, the present invention provides chromatographic means for separating from a starting lipid mixture a mixture that is enriched in docosapentaenoic acid relative to the starting lipid mixture. The invention also provides pharmaceutical formulations and dietary supplements including this enriched lipid formulation, and methods of supplementing docosapentaenoic acid in a subject by administering to the subject the pharmaceutical formulation or the dietary supplement. The invention also provides a method of resolving from the starting lipid mixture a mixture enriched in docosahexaenoic acid and eicosapentaenoic acid and depleted in docosapentaenoic acid, and formulations including this mixture (e.g., pharmaceutical formulations and dietary supplements).

In various embodiments, the invention also provides formulations of PUFAs, which optionally include one or more pharmaceutically acceptable diluent, excipient or other pharmaceutically acceptable component.

In various embodiments, the invention provides methods of using PUFAs and formulations thereof to prevent, treat or otherwise ameliorate a disease or condition in a subject. The method includes administering to the subject a therapeutically effective amount of a PUFA.

In an exemplary embodiment, the invention provides methods of supplementing docosapentaenoic acid and eicosapentaenoic acid levels in a subject by administering a formulation of the invention to the subject. In various embodiments, the formulations of the invention are of use to lower triglyceride levels in the subject to whom the formulation is administered. In an exemplary embodiment, the method results in the subject having lower serum total cholesterol than the subject would have in the absence of such administering. In an exemplary embodiment, the method results in the subject having lower serum LDL than the subject would have in the absence of such administering.

In various embodiments, the invention provides methods of preventing, treating or otherwise ameliorating inflammation in a subject. In various embodiments, the inflammation is associated with one or more neurodegenerative disorder, e.g., Alzheimer's Disease, Parkinson's Disease or Huntington's Disease.

In various embodiments, the method of the invention results in the lowering of the concentration of TNF-α in the brain of a subject treated with PUFA.

In an exemplary embodiment, the method of the invention results in lowering brain inflammation, such as inflammation that correlates with cognitive decline. In various embodiments, the method of the invention results in lowering in brain tissue one or more of IL-8 or MCP-1 concentration in a subject to whom the PUFA is administered.

In an exemplary embodiment, the method of the invention results in lowering brain inflammation, such as inflammation that correlates with cognitive decline. In various embodiments, the method of the invention results in lowering in brain tissue one or more of IL-8, TIMP2 and TNF-α in a subject treated with PUFA concentration in the brain, e.g., Hippocampus, of a subject to whom the PUFA is administered.

In an exemplary embodiment, the invention provides a method of modulating serum glucose increase in a subject, including preventing, treating or otherwise ameliorating metabolic syndrome.

### II. Abbreviations

PUFA, “polyunsaturated fatty acid”; FFA, “free fatty acid”; DHA, “docosahexaenoic acid”; DPA, “Docosapentaenoic acid”; EPA, “Eicosapentaenoic acid”.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are intended to be defined as indicated below.

### III. Definitions

The following terms are used in the claims of the patent as filed and are intended to have their broadest meaning consistent with the requirements of law. Where alternative meanings are possible, the broadest meaning is intended. All words used in the claims are used in the normal, customary usage of grammar and the English language.

By “a fatty acid of the omega-3 group”, is meant a polyunsaturated fatty acid, i.e. having at least one double bond, and for which the first double bond binds the carbon atoms 3 and 4 from the end chain. As an indication, the fatty acid may include 3, 4 or 5 double bonds. The fatty acid of the omega-3 group may include 18, 20, 22 or 24 carbon atoms. For example, the fatty acid of the omega-3 group may be an alpha-linolenic acid (ALA; C18:3), a stearidonic acid (SA: C18:4), an eicosatetraenoic acid (ETA; C20:4), an eicosapentaenoic acid (EPA; C20:5), a docosapentaenoic acid (DPA; C22:5), or a mixture of at least two of these compounds.

In the sense of the present invention, by “a food or health diet composition” is meant any type of product intended to be ingested by animal, notably human, organisms, which contain a fatty acid or a fatty acid mixture prepared by a method of the invention. Food supplements notably enter the field of protection of the present invention. Food supplements are products to be ingested, as a supplement to current food, in order to compensate for insufficiency of daily intakes of certain compounds. The food or health diet composition of the invention may be in the form of granules, powder, in liquid form naturally or suspended or put into a solution. It may appear in a suitable form for addition to the food ration of an animal or to any other product forming a food supplement. As such, the composition according to the invention may be in a dry, pasty, semi-pasty liquid or semi-liquid form. For example, these may be food products, beverages, food supplements and nutraceutical products.

Among the food products intended for human beings, more particularly relevant to the present invention, mention may be made of oils, margarines and other fats, yoghurts, cheeses, notably fresh cheeses and derived products, fermented products, dairy products, bread, rusks, and other cereal products or derived therefrom (for example pasta), cakes and biscuits, meal substitutes, snacks in general, foods intended for children, babies and infants, creams, desserts, ice creams, chocolate bars, cereal bars, fruit-based compotes.

According to an embodiment and in accordance with the present invention, the composition is in a suitable form for addition to the food ration of an animal. By “animal”, is more particularly meant in addition to humans, livestock and notably grazing animals (notably cattle reared for meat, milk and other dairy products, cheese and leather; sheep reared for meat, wool and cheese; goats; pigs), rabbits, poultry (chickens, hens, turkeys, ducks, geese and other poultry) reared for their meats and derived products including eggs, aquatic animals (for example animals from marine farms, fish, shrimps, oysters and mussels), leisure animals and pets (notably horses, dogs, cats, pet birds, aquarium fish), laboratory animals (notably rats and mice).

In various embodiments, the invention provides a pharmaceutical formulation. By “pharmaceutical formulation” is notably but not exclusively meant formulations including a PUFA (e.g., a PUFA purified by a method of the invention) in solid, liquid, pasty, semi-pasty, semi-liquid form in a mixture with one or more pharmaceutically acceptable component. Pharmaceutical formulations of the invention optionally include one or more pharmaceutically acceptable diluent, carrier, vehicle, excipient, additive and the like. The formulation is contrived so as to be suitable for formulating a PUFA, preferably an effective amount of the PUFA, e.g., a therapeutically effective amount of the PUFA. The formulation is adapted to the desired administration route and to the nature of the desired dosage form. Exemplary pharmaceutical compositions of the invention include a PUFA or a mixture of PUFAs prepared by a method of the invention and they are available in any dosage forms suitable for administration. Said dosage forms may notably consist in: tablets, gelatin capsules, powders, granules, lyophilizates, drinkable solutes, syrups, suspensions and suppositories. This list is not exhaustive. The term of “tablet” designates any kinds of tablets and notably effervescent tablets, dispersible tablets and orodispersible tablets.

When the composition of the invention is in the form of a granule or tablet, it may be in a coated form in order to avoid enzymatic destruction which occurs at a certain pH, and at the same time so as to allow controlled release of the active compound in another portion of the digestive tract. The composition according to the invention may also be found as sustained release or controlled release tablets.

“Pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable” also includes compounds that are, within the scope of medical judgment, suitable for use in humans without causing undesirable biological effects such as undue toxicity, irritation, allergic response, and the like, for example.

“Pharmaceutically acceptable vehicle” refers to a diluent, adjuvant, excipient or carrier with which a compound of the invention is administered. In an exemplary embodiment, a fatty acid or fatty acid mixture prepared by a method of the invention is incorporated into a pharmaceutically acceptable vehicle.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

Exemplary antioxidants include, without limitation, tocopherol and its derivatives, and rosemary extract. When tocopherol is utilized, it is present in an amount of from about 100-1000 ppm (e.g., 400 ppm). Other art-recognized pharmaceutically acceptable antioxidants are of use in the formulations of the present invention. Quite surprisingly, it has been discovered that combined antioxidants have a synergistic effect with respect to preventing and/or retarding degradation of formulations of the invention. An exemplary antioxidant combination includes tocopherols (20-2000 ppm), carnosic acid (20-500 ppm), lecithin (50-4000 ppm), and ascorbyl palmitate (5-500 ppm). In an exemplary embodiment, a mixture of antioxidants, such as the exemplary mixture, is incorporated into a unit dosage formulation of the invention.

These antioxidants have different mechanisms of action. Tocopherols are considered natural antioxidants and act as electron donors (Bauernfeind and Cort 1974). However, it has been demonstrated that at high levels they can act as strong pro-oxidants (Evans et al. 2002, Jung and Min 1992). Rosemary extract contains carnosic acid carnosol, and rosmarinic acid, which are also known as phenolic acids. Phenolic acids act as antioxidants by trapping free radicals (Brewer 2011). Ascorbyl palmitate works as an oxygen scavenger (Cort 1974), an entirely different mechanism from tocopherols or rosemary extract. During the oxidative process studies have shown that tocopherols are degraded over time in oil demonstrating significant decreases in oil after 6 to 8 months of storage (Rastrelli et al., 2002; Wang et al., 2010). Ascorbyl palmitate has been shown to preserve and protect tocopherols from being lost thusly improving their effectiveness in protecting the oil through via a synergistic effect (Beddows et al., 2001).

The term “excipients” is conventionally known to mean carriers, diluents and/or vehicles used in formulating drug compositions effective for the desired use.

The terms “effective amount” or “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

As used herein, the terms “treat” and “prevent” as well as words stemming therefrom, do not necessarily imply 100% or complete treatment or prevention. Rather, there are varying degrees of treatment or prevention of which one of ordinary skill in the art recognizes as having a potential benefit or therapeutic effect. In this respect, the methods of the present invention can provide any amount of any level of treatment or prevention of a disease or medical condition in a mammal. Furthermore, the treatment or prevention provided by the method can include treatment or prevention of one or more conditions or symptoms of the disease or medical condition. For example, with regard to methods of treating inflammation, the method in some embodiments, achieves a decrease in TNF-α levels in a subject. Also, for purposes herein, “prevention” can encompass delaying the onset of the disease, or a symptom or condition thereof. The term “treating” optionally includes prophylaxis of the specific disorder or condition, or alleviation of the symptoms associated with a specific disorder or condition and/or preventing or eliminating said symptoms. For example, as used herein the term “treating hypercholesterolemia” refers in general to altering cholesterol blood levels in the direction of normal levels and may include increasing or decreasing blood cholesterol levels depending on a given situation.

The phrases “active ingredient”, “therapeutic agent”, “active”, or “active agent” mean a chemical entity which can be effective in treating a targeted disorder, disease or condition.

The phrase “oral dosage form” means any pharmaceutical composition administered to a subject via the oral cavity. Exemplary oral dosage forms include tablets, capsules, films, powders, sachets, granules, solutions, solids, suspensions or as more than one distinct unit (e.g., granules, tablets, and/or capsules containing different actives) packaged together for co-administration, and other formulations known in the art. An oral dosage form can be one, two, three, four, five or six units. When the oral dosage form has multiple units, all of the units are contained within a single package, (e.g. a bottle or other form of packaging such as a blister pack). When the oral dosage form is a single unit, it may or may not be in a single package. In a preferred embodiment, the oral dosage form is one, two or three units. In a particularly preferred embodiment, the oral dosage form is one unit.

The phrase “unit”, as used herein, refers to the number of discrete objects to be administered which comprise the dosage form. In some embodiments, the dosage form includes a compound of the invention in one capsule. This is a single unit. In some embodiments, the dosage form includes a compound of the invention as part of a therapeutically effective dosage of a cream or ointment. This is also a single unit. In some embodiments, the dosage form includes a compound of the invention and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the invention in one capsule, and the active ingredient in a second capsule. This is a two unit dosage form, such as two capsules or tablets, and so such units are contained in a single package. Thus the term ‘unit’ refers to the object which is administered to the animal, not to the interior components of the object. In an exemplary embodiment, the unit dosage formulation of the invention is an oral unit dosage form. In various embodiments, the oral unit dosage form includes a therapeutically effective amount of a PUFA (e.g., prepared by a method of the invention), which is orally bioavailable.

Compounds produced by a method of the present invention may be esters (e.g., alkyl esters, mono-, di- or tri-glycerides, etc.), free acids or salts of free acids. Compounds produced by a method of the invention may contain relatively basic or acidic functionalities and salts of such compounds are included in the scope of the invention. Salts can be obtained by contacting the neutral form of such compounds with a sufficient amount of the desired acid or base, either neat or in a suitable inert solvent. Examples of salts for relative acidic compounds of the invention include sodium, potassium, calcium, ammonium, organic amino, or magnesium salts, or a similar salts. When compounds of the present invention contain relatively basic functionalities, acid addition salts can be obtained by contacting the neutral form of such compounds with a sufficient amount of the desired acid, either neat or in a suitable inert solvent. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science 1977, 66: 1-19). Certain specific compounds of the present invention contain both basic and acidic functionalities that allow the compounds to be converted into either base or acid addition salts. An exemplary salt is a “pharmaceutically acceptable salt”.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

### IV. Exemplary Embodiments

### Methods of Purification

In various embodiments, the present invention provides a novel method for enriching or isolating DPA from fish oil ethyl ester via RP (reverse phase)-HPLC. In an exemplary embodiment, the method of enriching or isolating DPA simultaneously provides one or more fractions enriched in EPA, DHA or a combination thereof.

In an exemplary embodiment, the invention provides a chromatographic method of resolving a lipid mixture of ω-3 polyunsaturated acids comprising eicosapentaenoic acid (C20:5n3), docosahexaenoic acid (C22:6n3) and docosapentaenoic acid (C22:5n3) into a first and second fraction.

In various embodiments, the method provides a first fraction comprising docosapentaenoic acid of purity from about 20% to about 90%, e.g., from about 40% to about 90%. In various embodiments, the invention provides a second fraction comprising a mixture of eicosapentaenoic acid (C20:5n3) in about 0% to about 90%, and docosahexaenoic acid (C22:6n3) in about 0% to about 90%, said method comprising submitting said mixture to a reverse-phase chromatographic separation.

In various embodiments, the method provides a first fraction comprising docosapentaenoic acid of a purity of at least about 75%. In an exemplary embodiment, this fraction comprises docosapentaenoic acid of a purity of at least about 90%.

In various embodiments, the first fraction comprising docosapentaenoic acid of purity of at least about 75% and a second fraction comprising a mixture of eicosapentaenoic acid (C20:5n3) in about 0% to about 90%, and docosahexaenoic acid (C22:6n3) in about 0% to about 90%, said method comprising submitting said mixture to a reverse-phase chromatographic separation, with the proviso that at least one of docosahexaenoic acid and eicosapentaenoic acid must be present in the second fraction in an amount greater than 0%.

In an exemplary embodiment, the method of the invention provides a reverse phase high pressure chromatographic method of enriching the amount of DPA in a mixture ω-3 polyunsaturated acids by at least about 5-, 6-, 7-, 8-, 9-, 10-, 11- 12-, 13- 14- or 15-fold relative to the amount of DPA in a starting mixture of ω-3 polyunsaturated acids.

In an exemplary embodiment, the method of the invention provides a reverse phase high pressure chromatographic method of enriching the amount of DHA relative to the amount of EPA in a mixture ω-3 polyunsaturated acids by at least about 1.2-, 1.3-, 1.4-, or 1.5-fold relative to the amount of DHA relative to the amount of EPA in a starting mixture of ω-3 polyunsaturated acids.

In various embodiments, the fatty acids are in ester form, e.g., alkyl ester, e.g., C₁-C₆ alky ester, e.g., ethyl ester.

In an exemplary embodiment, the compositions provided by the invention are not subjected to distillation, nor are they a product of distillation.

In an exemplary embodiment, the second fraction comprises ω-3 polyunsaturated acids other than eicosapentaenoic acid and docosahexaenoic acid in an amount of not more than about 10%, not more than about 5% or not more than about 1%.

In various embodiments, the reverse-phase chromatographic separation utilizes an eluent selected from an organic solvent and an eluent mixture of an organic solvent and water.

Various organic solvents can be used in practicing the reverse-phase chromatographic methods of the invention. In an exemplary embodiment, the organic solvent is selected from alcohols, e.g., MeOH, EtOH and i-PrOH; hydrocarbons, e.g., hexanes, petroleum ether; ketones, e.g., acetone, methylethyl ketone; esters, e.g., ethyl acetate; halocarbons, e.g., chloroform, methylene chloride; ethers, e.g., diethyl ether; tetrahydrofuran; aromatics, e.g., toluene; and a mixture thereof. In various embodiments, the organic solvent is selected from methanol and acetonitrile.

Amongst the advantages of the present invention is the ease with which commercially relevant amounts of the first and second fractions can be prepared. In an exemplary embodiment, the invention provides a reverse-phase chromatographic separation capable of resolving into the first fraction and the second fraction an amount of not less than about 10 metric tons of the starting lipid mixture per year.

In various embodiments, the invention accomplishes this goal using an efficient one-step separation cycle of large amounts of starting lipid mixture. In an exemplary embodiment, the reverse-phase chromatographic separation is capable of resolving into the first fraction and the second fraction an amount at least about 0.1 kg of the starting lipid mixture per separation cycle. In various embodiments, the chromatographic separation consists of a single separation cycle.

Starting lipid mixture from any animal, vegetable or other source can be used as the feedstock for the method of the invention. In an exemplary embodiment, the starting lipid mixture is derived from fish.

Following purification, the purified compounds can be derivatized as desired. In an exemplary embodiment, the compounds purified are simple alkyl esters of the fatty acids (e.g., methyl, ethyl or propyl esters). The esters can be saponified and, optionally, converted to another ester derivative such as a glyceride. In various embodiments, the esters are converted to different ester species by transesterification. In various embodiments, a fatty acid purified by a method of the invention is a component of a mono-, di- or tri-glyceride. In an exemplary embodiment, at least one of the remaining glycerol OH moieties is esterified with one acid selected from a short-, mid- or long-chain fatty acid and a phosphoric acid. Preferably, the glycerol ester is a triglyceride. In an exemplary embodiment, each fatty acid component of the glyceride is the same fatty acid. In various embodiments, a compound purified by a method of the invention is incorporated into a phospholipid, e.g., a ganglioside. Appropriate methods to saponify, esterify and transesterify compounds purified by methods of the invention are known and readily accessible to those of skill in the art.

In various embodiments, the free acids themselves, or salts thereof, are purified.

### Compositions—Pharmaceutical/Nutriceutical Formulations

In addition to the methods discussed herein, the invention also provides various compositions and formulations incorporating the first or second fraction prepared by the method of the invention. Exemplary formulations include those of use for incorporation into pharmaceutical formulations and dietary supplement formulations.

In various embodiments, the invention provides a pharmaceutical or dietary supplement formulation comprising the docosapentaenoic acid prepared by the chromatographic method according to the method of the invention. In an exemplary embodiment, this formulation comprises the eicosapentaenoic acid and docosahexaenoic acid mixture prepared by the chromatographic method of the invention.

In an exemplary embodiment, the invention provides a pharmaceutical formulation comprising docosapentaenoic acid, e.g., prepared by a chromatographic method according to the invention. In an exemplary embodiment, the docosapentaenoic acid is at least about 90%, at least about 91%, at least about 92%, at least about 93%, at least about 94%, at least about 95%, or at least about 96% pure.

In an exemplary embodiment, the invention provides a pharmaceutical formulation comprising docosahexaenoic acid, e.g., prepared by a chromatographic method according to the invention. In an exemplary embodiment, the docosahexaenoic acid is at least about 90%, at least about 91%, at least about 92%, at least about 93%, at least about 94%, at least about 95%, or at least about 96% pure.

In an exemplary embodiment, the invention provides a pharmaceutical formulation comprising eicosapentaenoic acid, e.g., prepared by a chromatographic method according to the invention. In an exemplary embodiment, the eicosapentaenoic acid is at least about 90%, at least about 91%, at least about 92%, at least about 93%, at least about 94%, at least about 95%, or at least about 96% pure.

In an exemplary embodiment, the invention provides a unit dosage pharmaceutical formulation comprising docosapentaenoic acid, e.g., prepared by a chromatographic method according to the invention. In an exemplary embodiment, the docosapentaenoic acid is at least about 90%, at least about 91%, at least about 92%, at least about 93%, at least about 94%, at least about 95%, or at least about 96% pure. In an exemplary embodiment, the unit dosage formulation includes the docosapentaenoic acid in an amount of from about 100 mg to about 5000 mg, e.g., from about 300 mg to about 3000 mg, e.g., from about 500 mg to about 1500 mg.

In an exemplary embodiment, the invention provides a unit dosage pharmaceutical formulation comprising docosahexaenoic acid, e.g., prepared by a chromatographic method according to the invention. In an exemplary embodiment, the docosahexaenoic acid is at least about 90%, at least about 91%, at least about 92%, at least about 93%, at least about 94%, at least about 95%, or at least about 96% pure. In an exemplary embodiment, the unit dosage formulation includes the docosahexaenoic acid in an amount of from about 100 mg to about 5000 mg, e.g, from about 300 mg to about 3000 mg, e.g., from about 500 mg to about 1500 mg.

In an exemplary embodiment, the invention provides a unit dosage pharmaceutical formulation comprising eicosapentaenoic acid, e.g., prepared by a chromatographic method according to the invention. In an exemplary embodiment, the eicosapentaenoic acid is at least about 90%, at least about 91%, at least about 92%, at least about 93%, at least about 94%, at least about 95%, or at least about 96% pure. In an exemplary embodiment, the unit dosage formulation includes the eicosapentaenoic acid in an amount of from about 100 mg to about 5000 mg, e.g., from about 300 mg to about 3000 mg, e.g., from about 500 mg to about 1500 mg.

In an exemplary embodiment, the invention provides a pharmaceutical formulation (Formulation I) including EPA, DHA and DPA. In an exemplary formulation, the DHA is the major component, and EPA and/or DPA are (is) present in a lesser amount than DHA. In an exemplary formulation, the DHA is the major component and EPA is present in an amount of from about 10% to about 20% of the amount of DHA in the formulation. In an exemplary formulation, the DHA is the major component and DPA is present in an amount of from about 8% to about 40% (e.g., from about 18% to about 30%) of the amount of DHA in the formulation. In an exemplary formulation, the DHA is the major component and EPA and DPA are present in an amount of from about 5% to about 30% (e.g., from about 10% to about 20%), and from about 8% to about 40% (e.g., from about 18% to about 30%), respectively, of the amount of DHA in the formulation. In an exemplary embodiment, this formulation is a unit dosage formulation.

In an exemplary embodiment, the invention provides a pharmaceutical formulation including EPA, DHA and DPA including from about 1% to about 20% EPA (e.g., from about 3% to about 13%); from about 42% to about 62% DHA (e.g., from about 52% to about 62% and from about 1% to about 30% DPA (e.g., from about 8% to about 18%). In an exemplary embodiment, this formulation is a unit dosage formulation.

An exemplary unit dosage formulation of the invention includes the first fraction prepared by a chromatographic method according to the invention. In various embodiments, the formulation includes an amount of docosapentaenoic acid sufficient to reduce triglyceride levels in a patient to whom the formulation is administered. In an exemplary embodiment, the docosapentaenoic acid is present in an amount of from about 100 mg to about 5000 mg. In various embodiments, the unit dosage formulation includes from about 100 mg to about 5000 mg of the first fraction produced by the method of the invention. In various embodiments, the unit dosage formulation includes an amount of the first fraction produced by the method of the invention sufficient to provide a formulation comprising from about 100 mg to about 5000 mg of docosapentaenoic acid.

In an exemplary embodiment, the invention provides a unit dosage pharmaceutical formulation comprising the second fraction produced by the chromatographic method of the invention. In this embodiment, the formulation includes a combination of eicosapentaenoic acid and docosahexaenoic prepared by a chromatographic method according to the invention. In various embodiments, the formulation includes an amount of the combination eicosapentaenoic acid and docosahexaenoic sufficient to reduce triglyceride levels in a patient to whom the formulation is administered. In an exemplary embodiment, the combination of eicosapentaenoic acid and the docosahexaenoic acid are present in an amount of from about 100 mg to about 5000 mg. In various embodiments, the unit dosage formulation includes from about 100 mg to about 5000 mg of the second fraction produced by the method of the invention. In various embodiments, the unit dosage formulation includes an amount of the second fraction produced by the method of the invention sufficient to provide a formulation comprising from about 100 mg to about 5000 mg of the combination of eicosapentaenoic acid and docosahexaenoic acid.

In various embodiments, the formulation of the second fraction includes a mixture of eicosapentaenoic acid and docosahexaenoic acid further comprising ω-3 polyunsaturated acids other than eicosapentaenoic acid and docosahexaenoic acid in an amount of not more than about 10%.

In various embodiments, the composition of the invention are delivered in a pill form, often with instructions for use, and often in the form of one or more pills that may deliver between 300 mg and 1000 mg of total oil per pill, of which often between 25% to 100% of this oil may be the various forms of omega-3 fatty acids discussed previously. Various other forms of delivery of a composition of the invention to a subject in need thereof may also be used. In exemplary embodiments, the composition is delivered as a bottled oil food or supplement to be taken by spoonful. In alternative formulations, the nutritional supplement is blended into other food products (e.g., peanut butter, margarine, salad oil, various drinks, and the like). In other formulations, the nutritional supplement is incorporated into various solid foods, or even delivered in a formulation suitable for enteric tube feeding or intravenous administration.

In each of the embodiments above in which a composition is described as an acid, it is understood that the acid is optionally in a salt or ester form. In an exemplary embodiment, the compounds are simple alkyl esters of the fatty acids (e.g., methyl, ethyl or propyl esters). In various embodiments, the fatty acid is a component of a mono-, di- or tri-glyceride. In an exemplary embodiment, at least one of the remaining glycerol OH moieties is esterified with one acid selected from a short-, mid- or long-chain fatty acid and a phosphoric acid. Preferably, the glycerol ester is a triglyceride. In an exemplary embodiment, each fatty acid component of the glyceride is the same fatty acid. In various embodiments, the fatty acid is incorporated into a phospholipid, e.g., a ganglioside. Appropriate methods to saponify, esterify and transesterify fatty acids are known and readily accessible to those of skill in the art.

### Methods of Treatment

The invention also provides various methods to provide supplementation of a PUFA to a subject in need thereof. In an exemplary embodiment, the method of supplementation includes administering docosapentaenoic acid, eicosapentaenoic acid and/or docosahexaenoic acid to the subject. In an exemplary embodiment, the invention includes administering to the subject a formulation of the invention comprising an amount of the first fraction effective to supplement the docosapentaenoic acid level in the subject. In various embodiments, the invention includes administering to the subject an amount of a formulation of the invention comprising the second fraction effective to supplement the eicosapentaenoic acid and docosahexaenoic acid levels in the subject.

In various embodiments, the method of the invention utilizes a formulation of the second fraction including a mixture of eicosapentaenoic acid and docosahexaenoic acid further comprising ω-3 polyunsaturated acids other than eicosapentaenoic acid and docosahexaenoic acid in an amount of not more than about 10%.

In various embodiments, the unit dosage formulation discussed hereinabove includes an amount of PUFA, e.g., prepared by a method of the invention, sufficient to prevent or treat a disease or condition in a subject in need of such prevention or treatment. In exemplary embodiments, the disease or condition is selected from dyslipodemia (e.g., high cholesterol), inflammation, high serum glucose, metabolic syndrome, diabetes, insulin resistance and neurodegenerative disease (e.g., Alzheimer's, Parkinson's and Huntington's Disease).

In an exemplary embodiment, Formulation I is administered to a subject, thereby lowering total serum cholesterol of the subject. In an exemplary embodiment, Formula I is administered to a subject to lower serum cholesterol of the subject. As is apparent from the examples appended hereto, Formulation I is more effective at lowering serum cholesterol concentration in a mammal than DHA, DPA or EPA or Lovaza®, a commercially available product containing a high percentage of EPA (46.5%) and 37.5% DHA. In an exemplary embodiment, this method is of use to prevent or treat dyslipidemia in the subject. In an exemplary embodiment, Formula I is administered as a unit dosage.

In an exemplary embodiment, Formulation I is administered to a subject, thereby lowering serum glucose concentration of the subject. In an exemplary embodiment, Formula I is administered to a subject to lower serum glucose concentration of the subject. As is apparent from the examples appended hereto, Formulation I is more effective than 95% DHA, 95% EPA or Lovaza®, and is approximately as effective as 95% DPA at modulating serum glucose concentration in a mammal to whom it is administered. In an exemplary embodiment, the method is of use to prevent or treat diabetes, metabolic syndrome and to regulate serum glucose levels in the subject to whom it is administered. In an exemplary embodiment, Formula I is administered as a unit dosage.

In an exemplary embodiment, Formulation I is administered to a subject, thereby lowering TNF-α concentration (pg/μg protein) of the subject. In an exemplary embodiment, Formula I is administered to a subject to lower TNF-α concentration (pg/μg protein) of the subject. As is apparent from the examples appended hereto, Formula I is more effective than 95% DPA, 95% EPA, 95% DHA or Lovaza® at lowering TNF-α concentration (pg/μg protein) in a mammal to whom it is administered. In an exemplary embodiment, the method is of use to prevent or treat a disease in which high levels of TNF-α concentration (pg/μg protein) are expressed in the subject to whom it is administered. In an exemplary embodiment, Formula I is administered as a unit dosage.

In an exemplary embodiment, Formulation I is administered to a subject, thereby lowering concentration in a subject of a member selected from IL-8, MCP-1 and a combination thereof to whom it is administered. In an exemplary embodiment, Formula I is administered to a subject to lower concentration in the subject of a member selected from IL-8, MCP-1 and a combination thereof. As is apparent from the examples appended hereto, Formula I is more effective than 95% DPA, 95% EPA 95% DHA or Lovaza® at lowering concentration in a mammal to whom it is administered of a member selected from IL-8, MCP-1 and a combination thereof. In an exemplary embodiment, administration of Formula I lowers IL-8 and MCP-1 in the brain or other nervous system tissue of the subject to whom it is administered. In an exemplary embodiment, the method is of use to prevent or treat inflammation and diseases in which inflammation is a component in the subject to whom it is administered. In an exemplary embodiment, Formula I is administered as a unit dosage.

In an exemplary embodiment, Formulation I is administered to a subject, thereby lowering concentration in the subject (e.g., in the hippocampus) of a member selected from IL-8, TIMP2, TNF-α and a combination thereof. In an exemplary embodiment, Formula I is administered to a subject to prevent or treat a neurodegenerative disease in the subject. As is apparent from the examples appended hereto, Formula I is more effective than 95% DPA, 95% EPA, 95% DHA or Lovaza® at lowering concentration in the subject (e.g., in brain, e.g., hippocampus, or other nervous system tissue) of a member selected from IL-8, TIMP2, TNF-α and a combination thereof to whom it is administered. In an exemplary embodiment, the method is of use to prevent or treat a disease selected from Alzheimer's Disease, Parkinson's Disease and Huntington's Disease in the subject to whom it is administered. In an exemplary embodiment, Formula I is administered as a unit dosage.

The optimal levels of omega-3 acids, esters and phospholipid forms of the fatty acids may be determined by various means, including animal studies. Here, for example, the methods of Corton et. al. (Journal of Biological Chemistry 279 (44), 46204-46212 (2004) may be used. Test animals such as mice, or even human subjects, may be fed a controlled diet containing various formulations of the nutritional supplement where the omega-3 fatty acids are set at various phospholipid to free fatty acid concentrations. The levels of gene expression (transcription) by the various lipid activated nuclear receptors, such as the PPAR-alpha receptors, may then be monitored using standard methods such as reverse transcriptase-PCR methods as detailed by Corton. These gene transcription levels, which may be considered to be one type of surrogate endpoint associated with life extension, can then be analyzed versus the omega-3 phospholipid to omega-3 free fatty acid composition of various nutritional supplement candidates, and the formulation associated with the highest level of gene expression, such as the highest level of PPAR-alpha activation, may be chosen.

In addition to looking directly at the transcription levels of certain genes associated with life extension, other markers of life extension may also be monitored, and the levels of omega-3 acid, ester or phospholipid associated with the desired effect (often the greatest effect at which unwanted side effects that do not also occur) may be chosen. These can be surrogate endpoints associated with life extension protocols such as caloric restriction, and can include endpoints or markers associated with reduced free T3 levels, reduced fasting serum insulin levels, reduced fasting serum leptin levels, reduced basal body temperature, reduced serum triglycerides, and enhanced beta fatty acid oxidation as indicated via a reduced respiratory quotient.

The following examples are intended to illustrate various embodiments of the invention. Because they are illustrative, they are not to be interpreted as limiting the scope of the invention in any manner.

### EXAMPLES

### Example 1

### Chromatographic Fractionation of PUFAs

### Materials

ΩmegaActiv® DPA 5000 ethyl esters were used as starting material (lot#12018-125D1D2EE). HPLC grade water and methanol were purchased from Alfa Aesar. EPA, DHA, and DPA ethyl ester standards were bought from Nu-Chek Prep, Inc.

### Semi-Preparative HPLC Method

HPLC System was Agilent 1100 (Agilent, Santa Clara, Calif., USA). The column used was YMC-Omega (Allentown, Pa., USA). The size of the column was 250×10 mm and it's packed with 50 μm particles with 120 Å pore size. Mobile phase was 100% methanol. The flow rate was 5.0 mL/min and injection volume was 100 μL. Column temperature was 25° C. Wavelength of detector was 220 nm.

### Preparation of Sample Using the Semi-Preparative HPLC

ΩmegaActiv® DPA 5000 (300 mg) was weighed individually into 7 injector vials and 1.0 mL of methanol was added to each vial to give a final concentration of 300 mg/mL. A total of 50 fractions were collected from 50 individual injections (100 μL each time) and the fractions were pooled into the same vessel. The methanol was removed from the pooled fractions via Rotovap (Buchi, Switzerland) at 425 mbar and 55° C. Once the pooled sample was dried to a constant weight, a stock solution of 25 mg/mL in methanol was made. This solution was diluted 1:250 to obtain a concentration of 0.1 mg/mL for analysis using analytical HPLC.

### Analytical HPLC Method for the DPA Enriched Ethyl Ester Sample

HPLC System was Jasco X-LC (Jasco, Easton, Md., USA). The column used was YMC-Triart C18 (YMC America, Allentown, Pa., USA). The size of the column was 50×2.0 mm and it was packed with 3 μm particles with 120 Å pore size. Mobile phase consists of a mixture of methanol and water (85:15, v:v). The flow rate was 0.2 mL/min and injection volume was 5 μL. Column temperature was 25° C. Wavelength of the detector was 220 nm.

Isolating DPA, EPA and DHA from ΩmegaActiv® DPA 5000

The same semi-preparative HPLC system and conditions were used. The ethyl ester sample was injected straight (no dilution with methanol) to the system at 100 μL. A total of 4 fractions were collected from 4 individual injections and the fractions were pooled into the same vessel. The methanol was removed from the pooled fractions via Rotovap (Buchi, Switzerland) at 300 mbar and 55° C. Once the pooled sample was dried to a constant weight, a stock solution of 5 mg/mL in methanol was made. This solution was diluted to obtain a concentration of 1.0 mg/mL for analysis. The analyses were done with the same method as described above.

### Results and Discussion

### Production of DPA Enriched Ethyl Ester Sample

DPA fraction was collected at 7.20 to 8.05 min from the semi-preparative HPLC system. The total injected ΩmegaActiv® DPA 5000 was 1516.6 mg and the fraction recovered was 151.2 mg. DPA content in starting material was determined to be 8.0% and in the final product DPA content was 77.9%. The recovery rate was calculated to be 97.05%. The RP-HPLC effectively recovered DPA with high concentration.

### Production of DPA, EPA and DHA Enriched Ethyl Ester Samples

In the starting material, the DPA, EPA, and DHA were determined by HPLC to be 8.23%, 4.59% and 48.54%, respectively. After injecting on to the semi-preparative RP-HPLC system, the First fraction was collected at 7.9 to 9.0 min, and the Second fraction was collected at 6.0 to 7.9 min. The First fraction weighed 31.77 g with a DPA content of 41.8% and the Second fraction weighed 241.08 g with an EPA content of 4.4%, and a DHA content of 65.12%. The total recovery for DPA, EPA, and DHA were 79.2%, 104.4%, and 92.1%, respectively.

### Example 2

### Materials and Methods

### Animal Tests

The hypotheses regarding the compositions of the invention were that 1) supplementing omega-3 fatty acids into the diets of low-density lipoprotein receptor null (LDLr−/−) mice would reduce total triglycerides and cholesterol in peripheral circulation, as well as reduce the accumulation of plaque in the aortic arch; 2) supplementation of purified DPAn3 (a single treatment from the first objective) would be more potent than EPA or DHA alone at attenuating inflammation and the accumulation of cholesterol-rich plaque in the aortic arch. Therefore our objectives were to 1) determine the influence of DPAn3 enrichment of macrophages on the inflammatory response in macrophages relative to SFAs, MUFA, and other PUFAs; 2) compare the effects of DPAn3, EPA, DHA, and commercial preparations of purified fatty acids (Lovaza® and ΩmegaActiv® DPA 5000) on the fatty acid composition of macrophages, lipid metabolism, and the development of atherosclerosis. Lovaza® was used as a comparison because it is an FDA approved fish oil supplement.

48 male LDLr−/− mice (8-10 weeks of age, ˜20 g) were obtained from Jackson Laboratories (stock #2207) and allowed to acclimate to the cages, room, and feeding design for 12 days. After the acclimation period mice were weighed and randomly assigned to one of six treatments. Mice were fed an isolipid (20% total fatty acids w/w), isonitrogenous, and 0.2% cholesterol (w/w) diet, which is considered atherogenic (Ain76, 58TQ TestDiet®) as a base diet. This base diet was formulated to meet or exceed all nutrient and energy requirements for a growing mouse. The treatments were added to the base diet (0.76% w/w of the total diet) and was made once at the beginning of the study, separated into weekly aliquots, stored at −80° C., allowed to thaw at 4° C. for 2 days prior to feeding, and stored at 4° C. until fed. A sample from each weekly aliquot was collected, composited, and archived for fatty acid analysis at a later date. The treatments were as follows: (1) Negative control diet, 3.5% of the total fatty acids were replaced with milk fat (the only source of fat in the base diet), (2) 3.5% of the total fatty acids were replaced with purified ethyl esters of DHA, (3) 3.5% of the total fatty acids were replaced with purified ethyl esters of DPAn3, (4) 3.5% of the total fatty acids were replaced with purified ethyl esters of EPA, (5) 3.5% of the total fatty acids were replaced with omega-3 fatty acids from Lovaza® (GlaxoSmithKline), and (6) 3.5% of the total fatty acids were replaced with omega-3 fatty acids from ΩmegaActiv® DPA 5000 (Omega Protein Inc., Houston, Tex.; Table 1).

TABLE 1
Basic composition of treatment diets fed to LDLr−/− mice.

| | | Control | DHA | DPAn3 | EPA | Lovaza ® | OmegaActiv |
|---|---|---|---|---|---|---|---|
| Protein | g/100 g diet | 17.6 | 17.6 | 17.6 | 17.6 | 17.6 | 17.6 |
| Carbohydrates | g/100 g diet | 50.2 | 50.2 | 50.2 | 50.2 | 50.2 | 50.2 |
| Linoleic Acid | g/100 g diet | 0.53 | 0.53 | 0.53 | 0.53 | 0.53 | 0.53 |
| Linolenic Acid | g/100 g diet | 0.1 | 0.1 | 0.1 | 0.1 | 0.1 | 0.1 |
| Milk Fat added | g/100 g Fat | 3.50 | 0 | 0 | 0 | 0 | 0 |
| to the diet | g/100 g diet | 0.76 | 0 | 0 | 0 | 0 | 0 |
| n-3 added to | g/100 g Fat | 0 | 3.50 | 3.50 | 3.50 | 3.50 | 3.50 |
| the diet | g/100 g diet | 0 | 0.76 | 0.76 | 0.76 | 0.76 | 0.76 |

All purified ethyl esters of fatty acids were purchased from NuCheck Prep, Inc. (Elysian, Minn.). Vitamin E and cholesterol levels were balanced across all the treatments. Mice were fed their diet three times weekly and intakes and refusals were recorded at these times. Daily feed intake was calculated and averaged across the whole time frame.

Mouse body weights were recorded once weekly in conjunction with cage changes. Blood serum lipid profiles were analyzed on blood samples from the mice before (subset of 6 mice), 10 weeks, and 20 weeks relative to initiation of dietary treatments. Three to five days prior to the end of the study each mouse was administered an intraperitoneal injection of 1.0 mL thiglycollate broth (4.05 g/dL) to elicit peritoneal macrophages. The peritoneal cells centrifuged then cultured in RPMI 1640 (+2 mM L-glutamine, +10 mM HEPES, +1 mM sodium pyruvate, +4500 mg/L glucose, +1500 mg/L sodium bicarbonate) on tissue culture plates (size) which allowed for adhesion of the macrophages. Plates where then vigorously washed to ensure proper macrophage isolation. Macrophages were scraped off the plates and aliquotted into a glass tube for fatty acid extraction, which was then performed via a modified Folch method. The heart and aorta were isolated from each mouse and immediately frozen in liquid nitrogen for later dissection. Aortic arches were dissected from the aortic root to the abdominal aorta using a microscope. Total lipid content was measured using the procedure outline by Folch, et al. (1957) J. Biol. Chem., 226:497-509, prior to cholesterol assessment. Total cholesterol, free cholesterol, and cholesterol ester were quantified as previously described (Wang, et al., 2010, Clin. Nutr., DOI: 10.1016/j.clnu.2013.04.009).

### Cell Culture Tests

THP-1 cells were maintained in RPMI 1640+2 mM L-Glutamine, 10 mM HEPES, 1 mM sodium pyruvate, 4500 mg/L glucose, and 1500 mg/L sodium bicarbonate; supplemented with 3.5 μL/L of 2-mercaptoethanol and 10% fetal bovine serum (FBS). Cell line was maintained in 75 cm² filtered cap flasks in a 37° C. incubator with 5% CO₂. THP-1 cells were seeded in 24 well plates at 5×105 cells/mL using 600 μL/well and allowed to differentiate into macrophages (MΦ) for 72 h with phorbol 12-myristate 13-acetate (PMA) at a final concentration of 50 ng/mL.

Experiment 1: Prior to incubation with individual fatty acids, the cells were starved for 8 h in a 2% lipo-free FBS RPMI medium. Individual fatty acids were added in duplicate into the 24 well plate at two concentrations, 50 μM and 100 μM. Every assay was cultured in three runs with duplicates in each run pooled. After incubation for 24 h with fatty acids the MΦ were scraped off the plates and fatty acids were isolated as described in the ‘Animal Tests’ portion of this Example. The fatty acid profiles of the MΦ were determined as previously described (Wang, et al., 2010, Clin. Nutr., DOI: 10.1016/j.clnu.2013.04.009).

Experiment 2: Previously differentiated MΦ were incubated with fatty acids as described in ‘Experiment 1’ for 24 hours. At the 24 h time point the cells were then stimulated with lipopolysaccharide from E. coli 0111:B4 for another 24 hours. The plates were centrifuged at 1200×g for 5 minutes and supernatant was then collected and analyzed for cytokines. The MΦ were scraped off the plates and fatty acids were isolated as described in the ‘Animal Portion’. The fatty acid profiles of the MΦ were determined as previously described (Wang, et al., 2010, Clin. Nutr., DOI: 10.1016/j.clnu.2013.04.009).

### Statistics

Average daily feed intake for the mice was cut into quartiles in order to assign a feed consumption class. A linear mixed effects model was used in R, lme in the nlme package, for estimating treatment effects on blood lipid profiles in mice. The fixed effects fit were treatment, and week of study, animal nested within treatment was fit as a random effect. Least squares means were calculated using the lsmeans package for all blood lipid profiles and a general linear hypothesis test, using glht in the multcomp package, was utilized for specified contrasts, control vs. the average of all omega-3 containing diets. Because Lovaza® is composed of high and almost equal levels of EPA and DHA we also contrasted the diet supplemented with Lovaza® to the average of the diets supplemented with EPA and DHA alone. Pairwise comparisons were made between time of blood analysis, and between treatments at each individual time frame.

An analysis of variance model was used in R, aov in the stats package, for estimating treatment effects on cholesterol deposition in mouse aortas. Least squares means and contrasts were analyzed as outlined above. Pairwise comparisons were made between treatments for TC, FC, and CE.

A linear mixed effects model was used in R, lme in the nlme package, for estimating treatment effects on cytokine production in LPS stimulated THP-1 cells. The fixed effects fit were treatment, FA concentration, and when significant LPS concentration was fit as a covariate, the plate cells were cultured on nested within run was fit as a random effect. Least squares means were analyzed as outlined above and pairwise comparisons were made between treatments for FA concentration, or where significant LPS concentration.

### Results and Discussion:

As expected, body weight and feed intakes increased over time, and there were no differences between treatments for body weight or daily food intake (P>0.10; Table 2); these results are in agreement with Wang, et al. (2013) Clin. Nutr., DOI: 10.1016/j.clnu.2013.04.009.

TABLE 2
Average feed intake, average daily gain, and percent lipid of aortas for LDLr−/− mice
fed an atherogenic diet supplemented (0.76% w/w of the diet) with milk fat (control)
or omega-3 fatty acids, alone or in combination.

| | Control | DHA | DPAn3 | EPA | Lovaza ® | OmegaActiv | P-Value |
|---|---|---|---|---|---|---|---|
| Feed intake, g/d | 3.95 | 3.91 | 3.78 | 3.88 | 3.94 | 4.01 | 0.74 |
| ADG, g/d | 0.17 | 0.17 | 0.18 | 0.16 | 0.17 | 0.17 | 0.84 |
| Aorta lipid, % | 29.55 | 36.38 | 29.52 | 29.93 | 38.80 | 30.71 | 0.62 |

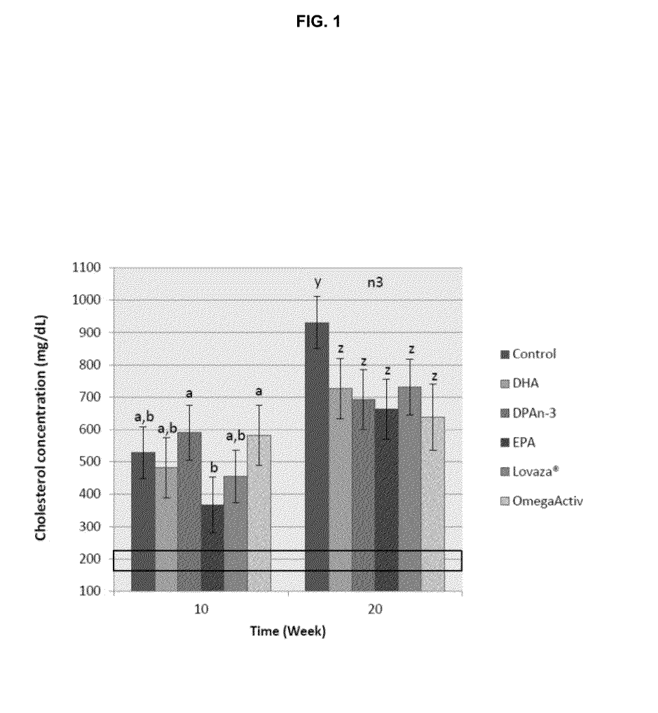

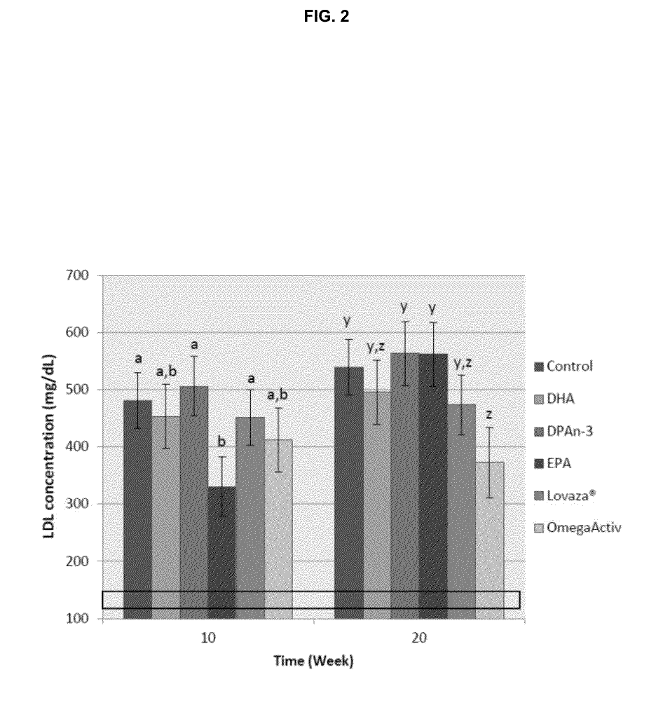

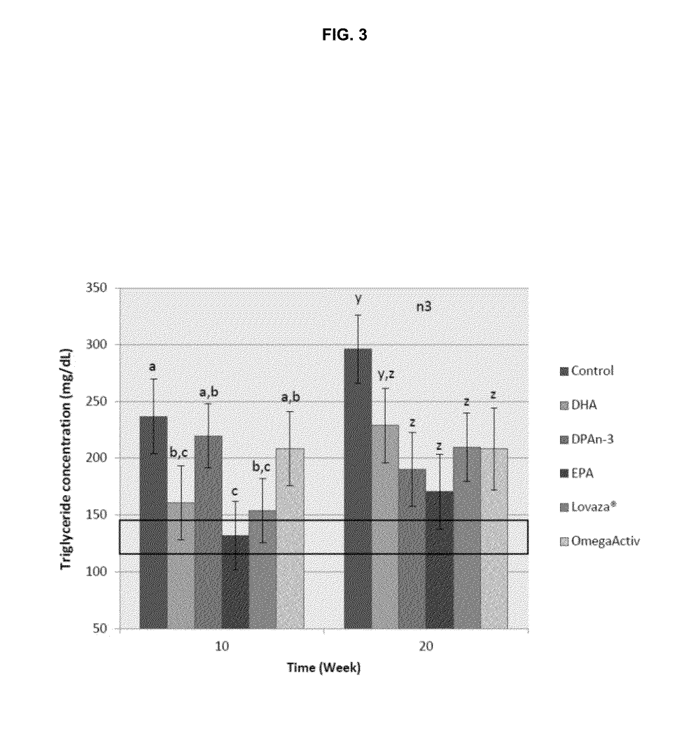

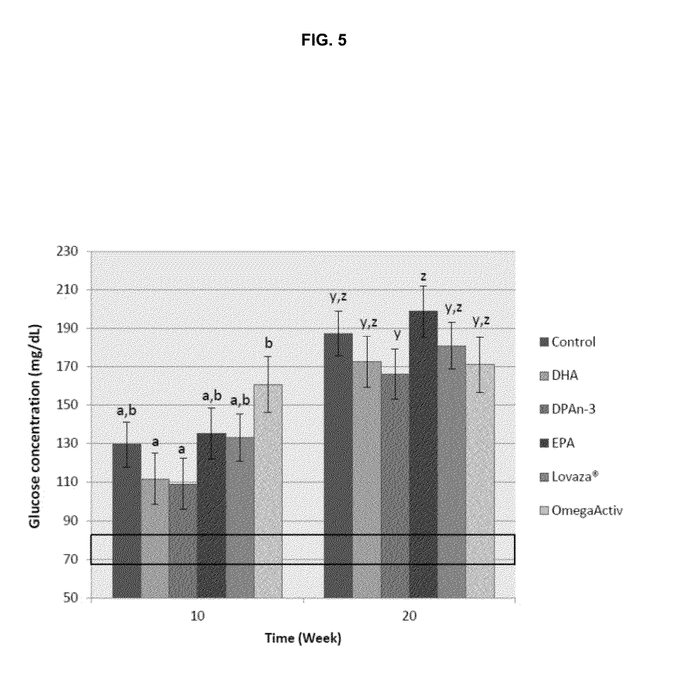

When leptin knockout mice were fed one of four diets, a control diet (9% fat, 46% sucrose, 20% casein), or the control diet with 11% of the fat replaced with EPA, DPAn3 or DHA for 4 weeks their food intakes did not differ; however, the mice supplemented with EPA gained the most weight (7.64 g) and the mice supplemented with DHA gained the least (Gotoh, et al., 2009, J. Agric. Food Chem., 57:11047-11054). Another study fed LDLr−/− mice a high-fat diet (23% calories from fat) as a control, and ApoE−/− mice a standard low-fat chow diet as another control. The treatments used the respective control diets (control diet) and supplemented with 1% w/w of fish oil (fish diet) or corn oil (corn diet). The mice were fed for 20 weeks and the mice on the fish diet had decreased feed intake compared to the mice on the corn diet or the control diet; but differences were not seen in body weight gains (Zampolli, et al., 2006, Atherosclerosis., 184:78-85). Total blood cholesterol, low-density lipoprotein (LDL) cholesterol, triacylglycerol (TAG), non-esterified fatty acid (NEFA), and glucose levels increased over the experiment period (FIGS. 1-5, respectively). These results are in agreement with Chen, et al., 2012 (Atherosclerosis, 221:397-404); indicating that these mice had dyslipidemia and insulin resistance. A serial harvest study was conducted on LDLr−/− mice from 0-12 months after feeding a high fat diet or a normal chow diet (Ma, et al., 2012, PLOS., 7:e35835.1-e35835.8), and they observed that total and LDL cholesterol concentrations in mice fed the high fat diet increased greatly after the first two weeks then slowly increased over the length of the study. They did not find an increase in total TAG level but did note an increase in fasting blood glucose concentrations.

Consistently high blood glucose concentrations over an extended period of time can increase the risk of developing insulin resistance. Supplementing a high fat diet (45% kcal from fat) with EPA (HF-EPA; 16% of total fatty acids) in C57BL/6J mice for 11 weeks did not prevent or reverse glucose tolerance as measured by area under the curve for a glucose tolerance test (Kalupahana, et al., 2010, J. Nutr., 140:1915-1922). However, when insulin resistance was assessed in this same study the HF-EPA diet prevented and reversed insulin resistance calculated as (glucose concentration*insulin concentration)/22.5 (homeostasis model of assessment of insulin resistance). The diet assessed as “reversing” was a high fat diet for the first 6 weeks of the study and the HF-EPA diet for the last 5 weeks of the study. Hyperglycemic LDLr−/− were found to not be insulin resistant as measured by injecting fed mice with 0.5 U/kg of body weight of human insulin and measuring blood glucose concentrations over time which was then expressed as a percentage of initial blood glucose concentration (Bonfleur, et al., 2010, Biochim Biophys Acta., 1801:183-190). After further investigation the pancreatic islets from LDLr−/− mice were less sensitive to stimulation by glucose than islets from C57BL/6J mice. Increased cholesterol deposition in pancreatic islet cells decreased insulin secretion in LDLr−/− mice, and when the cholesterol was depleted insulin secretion improved. The reduced pancreatic islet insulin secretion is the main cause of impaired glucose homeostasis in LDLr−/− mice (Bonfleur, et al., 2010, Biochim Biophys Acta., 1801:183-190). Excess circulating cholesterol, as is seen in LDLr−/− mice, causes cholesterol to be deposited in peripheral tissues instead of being taken up and recycled by the liver. Even though overall blood glucose concentrations increased throughout the present study, DPA treatment showed significantly lower glucose in blood than control and all other treatments.

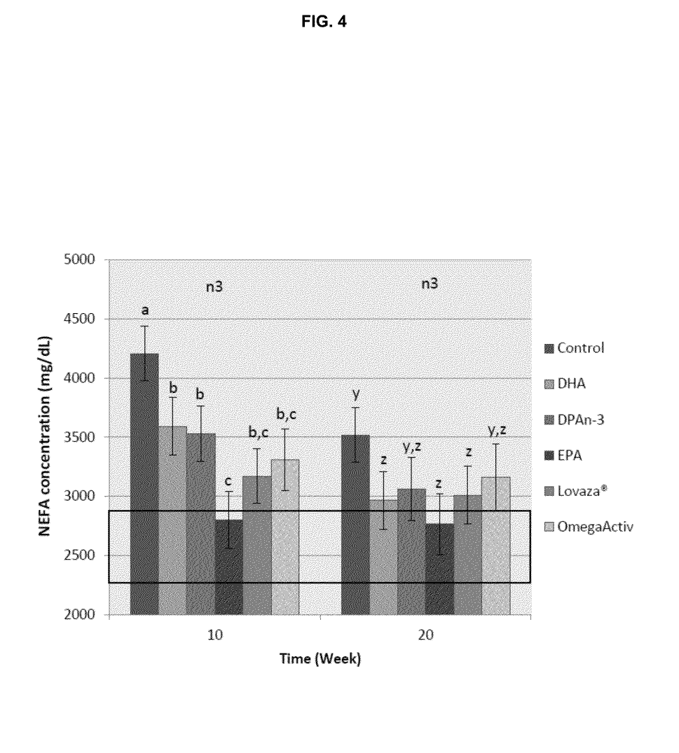

At the 10 and 20 week observations, NEFA concentrations among the mice on the Control diet were greater than the mice on diets containing omega 3 fatty acids (FIG. 4). In addition, at the 20 week observation, total blood cholesterol and TAG concentrations were greater among mice on the control diet than mice fed the omega 3 fatty acid diets; these results are in agreement with Gotoh, et al., 2009, J. Agric. Food Chem., 57:11047-11054; Wang, et al., 2009b., Atherosclerosis., 204:147-155; and Zampolli, et al. (2006), Atherosclerosis., 184:78-85, found increased total cholesterol as early as four weeks in LDLr−/− mice fed the control diet or the corn diet when compared to the fish diet and this trend continued to 20 weeks. They also observed increased TAG and LDL cholesterol at 20 weeks in the mice fed the control diet or the corn diet when compared to the fish diet. A high fat and cholesterol diet was fed to LDLr−/− mice for 14 weeks then the mice were switched to a normal chow diet with or without 5% EPA supplementation. This study found that the EPA supplementation decreased total cholesterol but not TAG. The discrepancy between the current study and that of in TAG concentration could be due to the mice in the Nakajima et al. (2011), Arterioscler. Thromb. Vasc. Biol., 31:1963-1972, study not consuming a high fat diet for 4 weeks at the time of blood lipid analysis. In the present study, the diet supplemented with Lovaza® was not different from the average of the diets supplemented with EPA and DHA alone for any of the serum lipids measured (P>0.10). Increased TAG and increased fasting glucose levels are two of five risk factors associated with increased risk of CVD and type 2 diabetes mellitus. The other three risk factors include increased weight circumference, decreased HDL, and increased blood pressure.

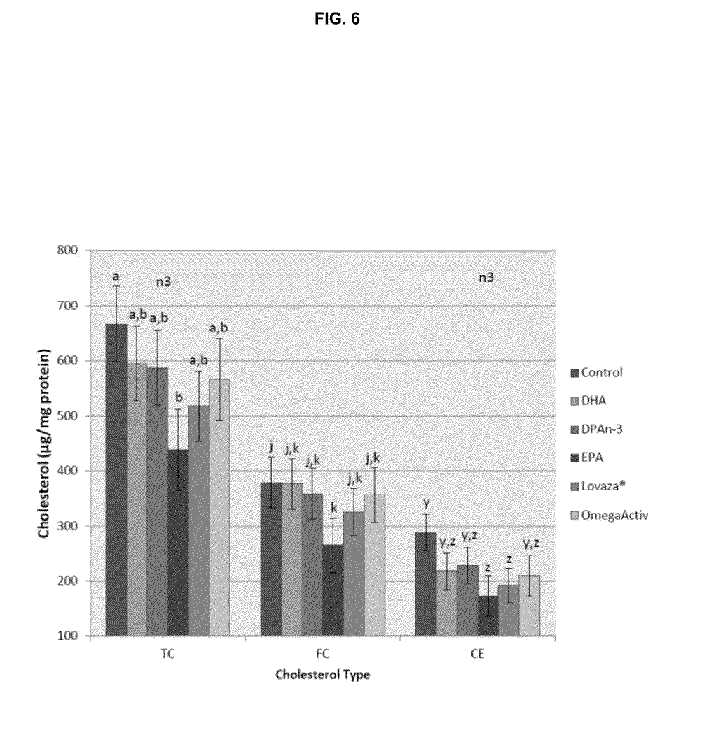

The percentages of total lipids in aortas were not different between treatments (P>0.10, Table 2). Total cholesterol (TC), free cholesterol (FC), and cholesterol ester (CE) accumulated in aortas among all mice (FIG. 6). All three measurements; TC, FC, and CE decreased with the addition of EPA to the diet (P<0.10). In addition, the diet containing Lovaza® decreased cholesterol ester deposition (P<0.05) when compared to the control diet. The average of all the omega-3 fatty acid diets when contrasted to the Control decreased TC and CE (P=0.1 and P<0.05, respectively). In the present study, the diet supplemented with Lovaza® was not different from the average of the diets supplemented with EPA and DHA alone for TC, FC, or CE (P>0.10). Plaque area size in transections of aortic tissue is another option to measuring cholesterol deposition in aortas, giving the ability to separate out the three main parts of the aorta: arch, descending, and infrarenal. Studies have found decreased plaque area in mice supplemented with fish oil (1 and 5 wt/wt), with the majority of the plaque being deposited in the aortic arch (Zampolli, et al., 2006, Atherosclerosis., 184:78-85; and Nakajima, et al., 2011, Arterioscler. Thromb. Vasc. Biol., 31:1963-1972, respectively). Interestingly it appears that after 9 months of feeding a high fat diet to LDLr−/− mice the aortic arch maxes out on lesion formation and at 12 months the lesions are much greater in the thoracic and abdominal aorta (descending), thus total artery lesion formation continues to increase (Ma, et al., 2012, PLOS., 7:e35835.1-e35835.8).

The build-up of plaque in the aortic wall is dependent on macrophage attraction, binding, and transfers from the lumen into the intima. Monocyte Chemoattractant Protein-1 (MCP-1) is an essential chemokine in the recruitment of monocytes and is secreted in response to oxidized LDL being taken up by resident macrophages. Crossing LDLr−/− mice with MCP-1−/− removes the ability of the LDLr−/− mouse to produce MCP-1. When the double knockout mouse was fed a high cholesterol diet atherosclerosis was severely inhibited, due to decreased macrophage presence in the aorta when compared to the LDLr−/− singular knockout mouse (Gu, et al., 1998, Mol. Cell., 2:275-281). Adhesion molecules play an important role in vascular invasion of monocytes to the intima of the aorta. A meta-analysis of randomized controlled trials researching the effects of n-3 PUFA supplementation on presence of adhesion molecules in plasma was conducted by Yang, et al. (2012), 95:972-980. They found omega-3 supplementation reduced plasma concentrations of sICAM-1, but not sVCAM-1, sP-Selectin, or sE-Selectin. Both endothelial cells and immune cells express ICAM-1, this includes monocytes, macrophages, and lymphocytes. VCAM-1 is expressed by cytokine stimulated endothelial cells that line large and small blood vessels (Gering, et al., 1993, Immunol. 14:506-512). The protective effect of omega-3 FAs was found in healthy individuals, and patients with dyslipidemia, and was attributed to inhibiting monocyte activation rather than endothelial activation (Yang et al., 2012, Am J Clin Nutr., 95:972-980).

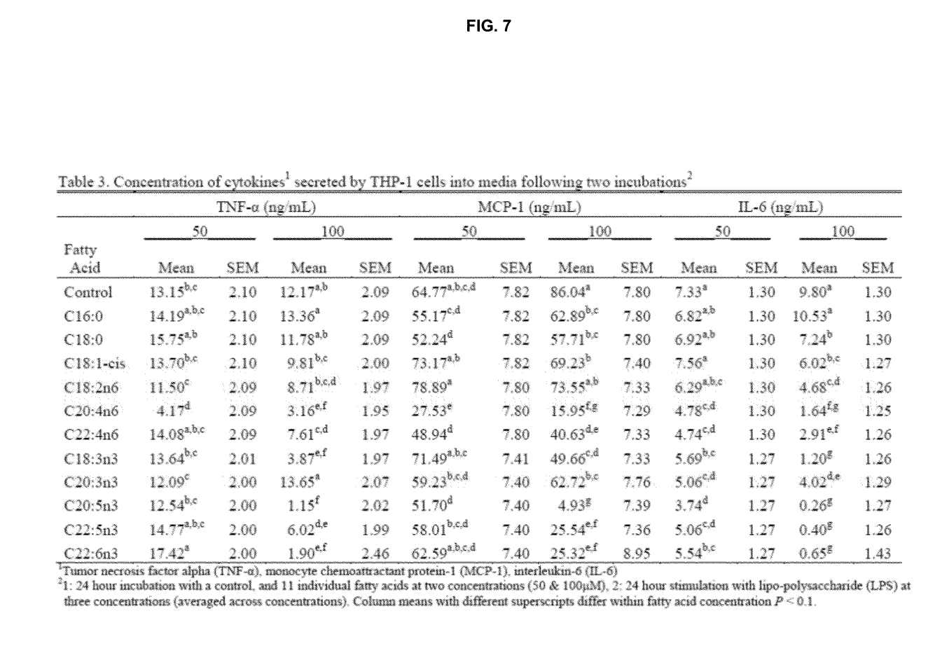

The type of fatty acid incubated altered (P<0.001) the release of THP-1 macrophage cytokines, TNF-α, MCP-1 and IL-6 (FIG. 7). Saturated fatty acids (C16:0 & C18:0) as well as oleic acid (C18:1-cis) did not change the amount of cytokines released by THP-1 cells when compared to the control macrophages, which received lipoprotein-free media with no supplemental fatty acids added. Incubating THP-1 cells with PUFAs decreased cytokine release, EPA was the most potent inhibitor of cytokine release followed by AA; these results are in agreement with Wang, et al., Br J Nutr., 102:497-501. Increasing the concentration of fatty acids altered the release of cytokines (P<0.01). Saturated fatty acids (C16:0 & C18:0) as well as oleic acid (C18:1-cis) did not change, or increased the amount of cytokines released by THP-1 cells when compared to the control macrophages. The inverse occurred when PUFAs were supplemented at increasing concentrations. When supplemented at the highest concentration of 100 μM EPA was the most potent inhibitor of cytokine release. When VLDL from mice supplemented with EPA (VLDL-E) was incubated with THP-1 cells cytokine release was attenuated compared to VLDL from mice on a normal chow diet (VLDL-C; Jinno et al., 2011, Atherosclerosis., 219:566-572). The authors found that VLDL-E was less susceptible than VLDL-C to lipoprotein lipase, this decreased free FA release and subsequent uptake by MΦ.

Arachidonic acid supplementation also decreased cytokine expression, but not to the extent of EPA supplementation. Hubbard et al. ((1993), J Leukoc. Biol., 54:105-110) found supplementing murine peritoneal macrophages with AA at concentrations as low as 1 μM decreased TNF-α production and completely inhibited it at 2-5 μM with increasing LPS concentrations having no effect on TNF-α inhibition. Arachidonic acid is present in the membrane of immune cells in relatively high amounts (generally 15-20%) when other 20 carbon fatty acids are not supplemented (Calder et al., 1990, Biochem. J., 269:807-814; and Fernandez et al., 1992). Arachidonic acid is also the preferred substrate for COX-1 and COX-2 resulting in the production of eicosanoids, which are produced in greater amounts post cellular stimulation (Calder, 2008, Prostaglandins. Leukot. Essent. Fatty Acids, 79:101-108). The addition of 50 μM AA to murine peritoneal MΦ culture increased the amount of PGE₂ present in the media regardless of LPS stimulation (Hubbard, et al., 1993, J Leukoc. Biol., 54:105-110). Without intending to be bound by any specific theory, this suggests that AA could be preferentially utilized by the COX pathways to produce eicosanoids, which may be having a negative feedback inhibition on pro-inflammatory cytokine production.

Omega-3 FAs are considered atheroprotective; in a controlled, nested, population based, case study a strong negative relationship (50% reduction in risk) was found between fish intake and risk for sudden death (Siscovick et al., 1995, JAMA, 274:1363-1367). The Physicians' Health Study reported an inverse relationship between circulating levels of long-chain omega-3 PUFAs and relative risk of sudden death in men with no medical history of CVD (Albert et al., 2002, N. Engl. J. Med., 346:1113-1118). The results of this study suggest that omega-3 FA can decrease risk factors associated with metabolic syndrome, aortic plaque buildup, and inflammation associated with endothelial damage and stress.

### Conclusions:

In this study omega-3 FA were shown to decrease plasma lipids and cholesterol deposition in the aortas of LDLr−/− mice. In addition, adding omega-3 FA to macrophage cell cultures decreased the secretion of pro-inflammatory cytokines after stimulation with LPS. Besides DPA, supplementing omega-3 FAs in the diets of LDLr−/− mice did not change fasting glucose concentrations in 20 weeks.

### Example 3

### EPA, DPA, and DHA Acids Decrease Macrophage Prostaglandin E2 and Inflammatory Cytokine Production

The objective of this study was to determine if docosapentaenoic acid n-3 (DPAn3) enrichment of macrophages (MΦ) changed their inflammatory response relative to saturated (S), mono-unsaturated (MU), and other poly-unsaturated (PU) fatty acids (FA). Differentiated THP-1 cells were incubated with one of 11 FA (50 and 100 μM) of varying degrees of unsaturation or no FA for 24 h prior to 24 h of stimulation with lipopolysaccharide from E. coli. Fatty acids were collected from MΦ without stimulation to determine the fatty acid profiles. Media was collected from MΦ post-stimulation and probed for prostaglandin E₂, and cytokines, including tumor necrosis factor-α, monocyte chemoattractant protein-1, and interleukin-6. Prostaglandin E₂ production was greater (P<0.05) for AA than all other FA, and omega-3 PUFA decreased (P<0.01) PGE₂ compared to n6 PUFA and all other FA. Incubating THP-1 cells with SFA, or MUFA did not change inflammatory cytokine release (P>0.10); however, PUFA decreased inflammatory cytokine release (P<0.01) and omega-3 PUFA were the most potent followed by arachidonic acid (AA). The results of this study suggest omega-3 PUFA can decrease inflammation associated with endothelial damage and stress. Further, PGE₂ production by THP-1 cells increased greatly when incubated with AA and likely has a negative feedback on inflammatory cytokine release.

### Example 4

### EPA, DPA, and DHA Fatty Acids Improve Serum Lipid Profiles and Decrease Aortic Plaque Buildup in LDLr−/− Mice Fed an Atherogenic Diet

Although the effects of eicosapentaenoic acid (EPA) and docosahexaenoic acid (DHA) on vascular disease markers have been well studied, the effect of docosapentaenoic acid omega-3 (DPAn3) has been studied less. The objective of this study was to compare the effects of DPAn3, EPA, DHA, and commercial combinations of EPA, DPAn3, and DHA on serum lipid profiles and the development of atherosclerosis in mice. Male LDLr−/− mice were assigned to one of six atherogenic (42% kcal from fat) dietary treatments (n=8) that had 3.5% of total fatty acids (FA) replaced with milk fat, DHA, DPAn3, EPA, or a commercial n3 fatty acid preparations. Blood samples were taken at 0, 10, and 20 weeks into the study; serum was isolated and probed for lipid classes and glucose. Aortic arches were harvested from all mice and total, free, and esterified cholesterol are reported. Blood lipid profiles and glucose in mice increased over the study, but supplementing omega-3 polyunsaturated fatty acids (PUFA) decreased (P<0.05) serum cholesterol, triglyceride, and non-esterified fatty acid concentrations compared to the milk fat control. Mice supplemented with all omega-3 PUFA had decreased aortic total cholesterol (P<0.10) and cholesterol ester (P<0.05) compared to the control mice. The results of this study suggest that EPA, DPA, and DHA can (P>0.10) improve serum lipid profiles and decrease aortic plaque buildup in a mouse model prone to atherosclerosis.

### Example 5

### The Effects of Omega-3 Docosapentaenoic Acid on Atherosclerosis, Brain Inflammation, and Macrophage Inflammatory Mediator Production

The objectives of these studies were [1] to compare the effects of replacing 3.5% of the total fatty acids with DPAn3, EPA, DHA, and commercial combinations of EPA, DPAn3, and DHA on serum lipid profiles, brain inflammation, and the development of atherosclerosis in LDLr−/− mice fed an atherogenic diet for 20 weeks and [2] to determine if DPAn3 enrichment of macrophages (50 and 100 μM) changed their lipopolysaccharide stimulated inflammatory profile relative to saturated (S), mono-unsaturated (MU), and other omega-6 and omega-3 poly-unsaturated (PU) fatty acids (FA). [1] Serum lipids increased in mice over the study, but supplementing omega-3 PUFA decreased (P<0.05) serum cholesterol, triglyceride, and non-esterified fatty acid concentrations compared to the milk fat control. All mice supplemented with omega-3 PUFA had decreased aortic total cholesterol (P<0.10) and cholesterol ester (P<0.05) compared to the control mice Immunohistochemistry analysis of the cerebrovasculature in omega-3 supplemented mice showed decreased (P<0.01) IL-6, TNF-α, thrombin, and vascular endothelial growth factor compared to controls. Similarly, omega-3 supplemented mice had decreased (P<0.05) expression of IL-6, IL-8, MCP-1, TNF-α, thrombin, thrombospondin-1, and TIMP-2 in the hippocampus region of the brain. Prostaglandin E₂ production was greater (P<0.05) for MΦ enriched with AA when compared to all other FA, and omega-3 PUFA decreased (P<0.01) PGE₂ compared to all other FA. Incubating THP-1 cells with SFA or oleic acid did not change inflammatory cytokine release (P>0.10). The PUFA decreased inflammatory cytokine release (P<0.01) and omega-3 PUFA were the most potent. These studies suggest that at moderately high concentrations, EPA, DPAn3, and DHA can (P>0.10) improve serum lipid profiles, decrease aortic plaque buildup in a mouse model prone to atherosclerosis, and decrease brain inflammation.

Examples 2-5 concluded that EPA, DHA, and DPA were shown to 1) decrease plasma lipids and cholesterol deposition in the aortas; 2) decrease inflammatory secretion; 3) improve serum lipid profile and decrease aortic plaque buildup; 4) decrease brain inflammation. In example 2, DPA was the only omega-3 showing lower fasting glucose level than control. Examples 6 to 10 showed the uniqueness of DPA and Formulation I (megaActiv® DPA 5000).

### Example 6

### DPA and ΩmegaActiv® DPA 5000 Inhibited the Increase of Total Cholesterol, LDL Cholesterol and Triglyceride in Mice Serum

Total cholesterol and LDL cholesterol levels in mice serum fed with different omega-3 products

| | Serum total | Serum LDL |
|---|---|---|
| | cholesterol (mg/dL) | cholesterol (mg/dL) |
| Control (week 0) | 200.00 | 140.00 |
| Control (week 20) | 931.13 | 539.94 |
| DHA (week 20) | 726.86 | 495.51 |
| DPA (week 20) | 692.98 | 563.45 |
| EPA (week 20) | 663.37 | 562.07 |
| Lovaza ® (week 20) | 731.47 | 474.53 |
| ΩmegaActiv ® DPA 5000 | 637.85 | 372.96 |
| (week 20) | | |

ΩmegaActiv® DPA 5000 fed mice had the lowest levels of total cholesterol and LDL cholesterol in serum.

### Example 7

### DPA is Most Effective on Inhibiting Serum Glucose Increase

Glucose concentration in serum of mice fed with different omega-3 products

| | Glucose concentration (mg/dL) |
|---|---|
| Control (week 0) | 74.00 |
| Control (week 20) | 187.39 |
| DHA (week 20) | 172.58 |
| DPA (week 20) | 166.23 |
| EPA (week 20) | 198.65 |
| Lovaza ® (week 20) | 181.00 |
| ΩmegaActiv ® DPA 5000 (week 20) | 171.01 |

DPA fed mice showed the lowest glucose concentration in serum.

### Example 8

### ΩmegaActiv® DPA 5000 and DPA Decreased the TNF-α Level in Mice Adipose Tissue

TNF-α concentration in adipose tissue of mice fed with different omega-3 products

| | TNF-α Concentration (pg/μg protein) |
|---|---|
| Control | 1.57 |
| DHA | 0.82 |
| DPA | 0.41 |
| EPA | 0.58 |
| Lovaza ® | 0.47 |
| ΩmegaActiv ® DPA 5000 | 0.30 |

Tumor necrosis factor-alpha (TNF-α) is a pleiotropic inflammatory cytokine. Although TNF-α causes necrosis of some types of tumors, it promotes the growth of other types of tumor cells. High levels of TNF-α correlate with increased risk of mortality. ΩmegaActiv® DPA 5000 treatment showed the lowest TNF-α level in mice adipose tissue.

### Example 9

### DPA and ΩmegaActiv® DPA 5000 Decreased Brain Inflammation Associated with Cognitive Decline

Flourescence intensity (relative) of brain microvessel

| | IL-8 | MCP-1 |
|---|---|---|
| Control | 1.00 | 1.00 |
| DHA | 0.86 | 0.93 |
| DPA | 0.96 | 0.92 |
| EPA | 0.93 | 0.96 |
| Lovaza ® | 0.90 | 0.85 |
| ΩmegaActiv ® DPA 5000 | 0.84 | 0.83 |

Interleukin 8 (IL-8) is a proinflammatory cytokines which can promote brain inflammation.

Monocyte chemoattractant protein-1 (MCP-1) is related with the recruitment of monocytes to sites of injury and infection. MCP-1 has been found in the joints of people with rheumatoid arthritis where may serve to recruit macrophages and perpetuate the inflammation in the joints.

ΩmegaActiv® DPA 5000 was most effective reducing brain inflammation cytokines IL-8 and MCP-1 in the brain microvessel.

### Example 10

### Flourescence Intensity (Relative) of Brain Hippocampus

| | IL-8 | TIMP2 | TNF-α |
|---|---|---|---|
| Control | 1 | 1 | 1 |
| DHA | 0.46 | 0.23 | 0.29 |
| DPA | 0.70 | 0.17 | 0.28 |
| EPA | 0.72 | 0.63 | 0.31 |
| Lovaza ® | 0.50 | 0.49 | 0.54 |
| ΩmegaActiv ® DPA 5000 | 0.24 | 0.54 | 0.10 |

TIMP metallopeptidase inhibitor 2 is a well-known angiogenesis inhibitor.

Angiogenesis is a vital process in growth and development, as well as in wound healing and the formation of granulation tissue.

TNF-α was found to be significantly higher in parkinsonian patients than normal people and it is believed to be related to neuronal degeneration.

DPA was most effective on inhibiting TIMP2 levels in brain Hippocampus. ΩmegaActiv® DPA 5000 was most effective reducing brain inflammation cytokines IL-8 and TNF-α in the brain Hippocampus.

### Example 11

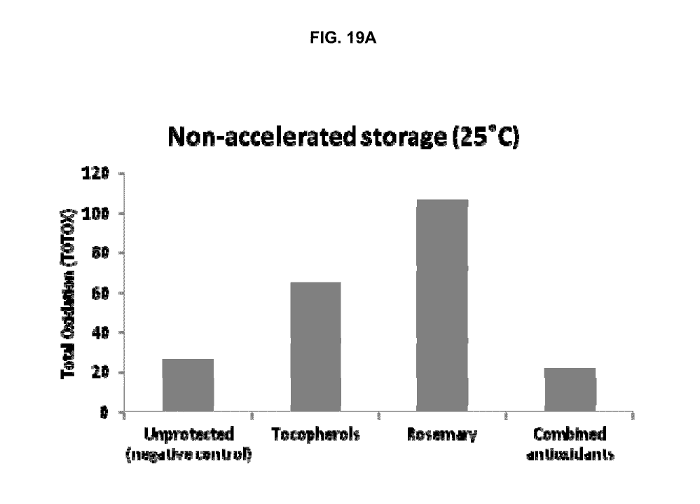

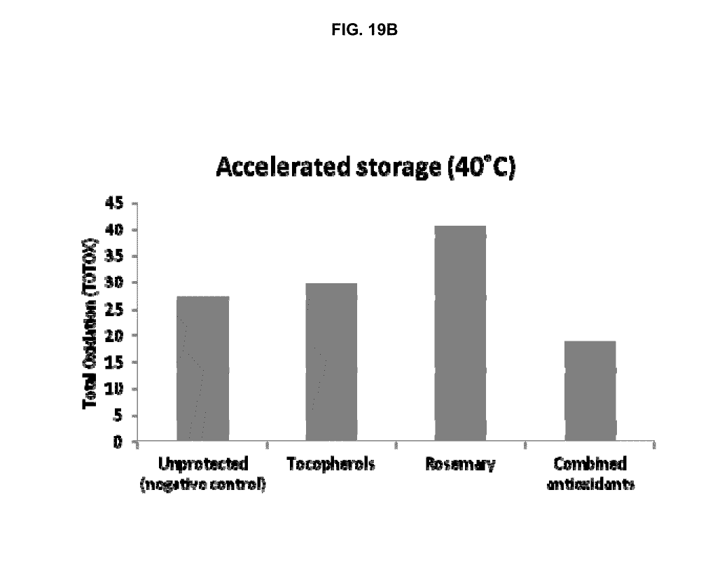

The non-accelerated (25° C.; FIG. 19) and accelerated (40° C.; FIG. 20) studies have been conducted on ethyl ester concentrates. Treatments included a Negative control, Combined antioxidants (a mixture of rosemary, tocopherols, ascorbic palmitate, and lecithin), 400 ppm of Tocopherols, and 5000 ppm rosemary extract. Conclusively, the mixture of antioxidants appears to provide the best protection in both accelerated and non-accelerated conditions.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

All publications, patents, and patent applications cited herein are hereby incorporated by reference in their entirety for all purposes.

What is claimed is:

1. A chromatographic method of resolving a lipid mixture of ω-3 polyunsaturated acids comprising eicosapentaenoic acid (C20:5n3), docosahexaenoic acid (C22:6n3) and docosapentaenoic acid (C22:5n3) into a first and second fraction, said first fraction comprising docosapentaenoic acid of purity from about 20% to about 90% and a second fraction comprising a mixture of eicosapentaenoic acid (C20:5n3) in about 0% to about 90%, and docosahexaenoic acid (C22:6n3) in about 0% to about 90%, said method comprising submitting said mixture to a reverse-phase high pressure liquid chromatography separation.

2. The method according to claim 1, wherein the second fraction comprises ω-3 polyunsaturated acids other than eicosapentaenoic acid and docosahexaenoic acid in an amount of not more than about 10%.

3. The method according to claim 1, wherein the first fraction comprises docosapentaenoic acid in an amount not less than about 90% of the formulation.

4. The method according to claim 1, wherein the reverse-phase chromatography separation utilizes an eluent selected from an organic solvent and an eluent mixture of an organic solvent and water.

5. The method of claim 4, wherein the organic solvent is selected from alcohol, hydrocarbons, ketones, esters, halocarbons, ethers, aromatic and/or a mixture thereof.

6. The method of claim 5, wherein the organic solvent is selected from methanol and acetonitrile.

7. The method of claim 1, wherein the ω-3 polyunsaturated acids of the lipid mixture of ω-3 polyunsaturated acids are in the alkyl ester form.

8. The method of claim 1, wherein the reverse-phase chromatography separation is capable of resolving into the first fraction and the second fraction an amount of said lipid mixture of ω-3 polyunsaturated acids of at least about 0.1 kg per separation cycle.

9. The method of claim 1, wherein the reverse-phase high pressure liquid chromatography separation consists of a single separation cycle.

10. The method of claim 1, wherein the lipid mixture is derived from fish.

11. A pharmaceutical formulation comprising a fraction prepared by the method of claim 1, said fraction selected from fraction 1 and fraction 2.

12. A unit dosage pharmaceutical formulation comprising a fraction prepared by the method of claim 1, said fraction selected from fraction 1 and fraction 2 in an amount of from about 100 mg to about 5000 mg.

13. The pharmaceutical formulation according to claim 11 comprising docosapentaenoic acid of purity not less that about 90%.

14. The pharmaceutical formulation according to claim 11, further comprising a pharmaceutically acceptable carrier.

15. The pharmaceutical formulation according to claim 14, wherein the pharmaceutical formulation is a unit dosage formulation.

16. The pharmaceutical formulation according to claim 15, wherein the unit dosage formulation comprises from about 100 mg to about 5000 mg of the docosapentaenoic acid.

17. The pharmaceutical formulation of claim 11, comprising a mixture of eicosapentaenoic acid and docosahexaenoic acid further comprising ω-3 polyunsaturated acids other than eicosapentaenoic acid and docosahexaenoic acid in an amount of not more than about 10%.

18. The pharmaceutical formulation according to 17, further comprising a pharmaceutically acceptable carrier.

19. The pharmaceutical formulation according to claim 17, wherein the pharmaceutical formulation is a unit dosage formulation.

20. The pharmaceutical formulation according to claim 19, wherein the unit dosage formulation comprises from about 100 mg to about 5000 mg of the eicosapentaenoic acid and from about 100 mg to about 5000 mg of the docosahexaenoic acid.

22. A method of supplementing the docosapentaenoic acid level in a subject in need of such supplementation, the method comprising administering to the subject an amount of the pharmaceutical formulation according to claim 11 effective to supplement the docosapentaenoic acid level in the subject.

23. The method of claim 22, wherein said subject has a condition selected from dyslipodemia (e.g., high cholesterol), inflammation, high serum glucose, metabolic syndrome, diabetes, insulin resistance cognitive decline, and neurodegenerative disease (e.g., Alzheimer's, Parkinson's and Huntington's Disease).

24. A method of supplementing the eicosapentaenoic acid and docosahexaenoic acid levels in a subject in need of such supplementation, the method comprising administering to the subject an amount of the pharmaceutical formulation according to claim 11 effective to supplement the eicosapentaenoic acid and docosahexaenoic acid levels in the subject.

25. The method of claim 24, wherein said subject has a condition selected from dyslipodemia (e.g., high cholesterol), inflammation, high serum glucose, metabolic syndrome, diabetes, insulin resistance cognitive decline, and neurodegenerative disease (e.g., Alzheimer's, Parkinson's and Huntington's Disease).
